(19) United States
(12) Patent Application Publication
(10) Pub. No.: US 2026/0325460 A1
(43) Pub. Date: Oct. 22, 2026

(54) COMPOSITIONS, METHODS AND USES FOR DETERMINING ATOMIC LAYER DEPOSITION COATING QUALITY AND DISSOLUTION RATES

(71) Applicant: VITRIVAX, INC., Boulder, CO (US)

(72) Inventors: Bryan L. STEADMAN, Boulder, CO (US); Lorena R.A. NAPOLITANO, Boulder, CO (US); Emily M. HITE, Boulder, CO (US)

(21) Appl. No.: 19/692,754

(22) Filed: May 29, 2026

Related U.S. Application Data
(63) Continuation of application No. PCT/US2024/058293, filed on Dec. 3, 2024.
(60) Provisional application No. 63/605,994, filed on Dec. 4, 2023.

(51) Int. Cl. A61K 9/51; A61K 9/50; A61K 45/06; G01N 25/02

(52) U.S. Cl. CPC A61K 9/5192; A61K 9/5073; A61K 9/5115; A61K 45/06; G01N 25/02

(57) ABSTRACT

Embodiments of the present disclosure provide novel compositions and methods for determining atomic layer deposition (ALD) coating quality and/or dissolution rates of ALD coated therapeutic agent containing particles. In certain embodiments, in vitro methods can be used to predict or project in vivo dissolution rates of ALD coated therapeutic agent-containing particles, microparticles and/or nanoparticles. In yet other embodiments, formulations disclosed herein can be used to provide improved conditions for determining ALD coating quality or composition, optimum coating layers and/or dissolution rates to release a target therapeutic agent from ALD coating layers. In accordance with these embodiments, improved coating compositions, coating cycles, coating layering and thickness can be determined for improved timed-release delivery of pharmaceutical products to a subject in need thereof.

### CROSS-REFERENCED APPLICATION

This Continuation Application claims priority to International Application No. PCT/US2024/058293, filed Dec. 3, 2024, which claims priority to U.S. Provisional Application No. 63/605,994, filed Dec. 4, 2023. These applications are incorporated herein by reference in their entireties for all purposes.

### FIELD

Embodiments of the present disclosure provide novel compositions and methods for determining atomic layer deposition (ALD) coating quality and/or dissolution rates. In certain embodiments, in vitro methods can be used to project or predict in vivo dissolution rates of ALD coated particles, microparticles and/or nanoparticles harboring or encasing at least one therapeutic agent. In yet other embodiments, formulations disclosed herein can be used to provide improved conditions for determining ALD coating quality and/or dissolution rates of ALD coated particles, microparticles and/or nanoparticles harboring or encasing at least one therapeutic agent. In accordance with these embodiments, improved coating compositions, coating cycles, coating layering and thickness can be determined for improved ALD-coating, storage and delivery of therapeutic agents of therapeutic agent-containing particles.

### BACKGROUND

Understanding dissolution of coated therapeutic agents can be critical in the success of delivery of the therapeutic agents to treat a targeted health condition or to reduce or prevent onset of a health condition. Designing and using coated particles that provide enhanced therapeutic outcomes is difficult due in part to the targeted therapeutic agent, coating compositions and the number of coating layers used to create a timed-release product. Currently, there are no reliable in vitro assays having an in vitro to in vivo correlation for assessing optimal coating of a therapeutic agent, for example, optimal ALD coating of a therapeutic agent.

### SUMMARY

Embodiments of the present disclosure provide novel compositions for analyzing dissolution rates of ALD coated therapeutic agent-containing particles and methods for determining ALD coating quality. In certain embodiments, in vitro methods can be used to determine in vivo dissolution rates of ALD coated therapeutic agent-containing particles, microparticles and/or nanoparticles. In some embodiments, formulations or compositions disclosed herein include agents for optimal analysis of dissolution rates of insoluble or highly insoluble ALD coated therapeutic agent-containing particles. In accordance with these embodiments, compositions disclosed herein can be used to provide for improved timed-release delivery of pharmaceutical or therapeutic products to a subject in need thereof.

In some embodiments and further to paragraph above, in vitro dissolution assays can be used to understand mechanisms and kinetics of release in vivo of a therapeutic agent of a targeted ALD-coated therapeutic agent-containing particle. In other embodiments, in vitro dissolution assays can be used to understand mechanisms and kinetics of release in vivo of a therapeutic agent of a targeted ALD-coated therapeutic agent-containing particle for quality control. In certain embodiments, in vitro dissolution assays disclosed herein are designed to assess dissolution of poorly soluble delayed release therapeutics. In certain embodiments, ALD coated therapeutic agent-containing particles designed as single shot, delayed release therapeutic agents' dissolution rates can be assessed by compositions and methods disclosed herein. In some embodiments, ALD coated therapeutic agent-containing particle dissolution rates can be assessed by compositions and methods disclosed herein.

In some embodiments and further to paragraphs [0004]-[0005] above, ALD coated therapeutic agent-containing particles disclosed herein can include metal oxide or metallo-organic material coated particles containing at least one thermostable antigen with a defined number of ALD coating cycles to enable release of the at least one thermostable antigen at a specified time, or times, post-administration. In certain embodiments, the metal oxide coating layers can include aluminum oxide, an aluminum alkoxide (e.g., alucone), silicon dioxide (SiO₂), titanium dioxide (TiO₂), zinc oxide (ZnO₂), zirconium oxide (ZrO₂) or silicon nitride (Si₃N₄) alone, in alternating layers, or other suitable layer pattern, or in a suitable combination composition for layering on thermostable therapeutic-agent containing particles contemplated herein. In some embodiments, the metal oxide coating layers can include aluminum oxide alone, in alternating layers, or in a combination layer composition with at least one additional agent (e.g., silicon dioxide).

In certain embodiments and further to paragraphs [0004]-[0006] above, the ALD applied coating layers to therapeutic agent containing particles contemplated herein can be about 0.1 nm to about 40 nm in thickness, about 0.1 nm to about 20 nm in thickness, or about 0.1 nm to about 10 nm in thickness. In some embodiments, thermostable therapeutic agent-containing particles can include coating layers sufficient to uniformly coat the particles to delay release or provide a timed-release of the at least one therapeutic or immunogenic agent from a central or innermost therapeutic or immunogenic agent-containing particle. In other embodiments, a coat of desired thickness relative to thermostable particles (e.g., spray dried particles) can include a defined number of ALD coating cycles to enable release of therapeutic agents, immunogenic agents and/or antigen(s) at a specified time, or times, post-administration. Because the time-to-release can be anywhere from days to months, it can be challenging to model dissolution of these ALD coated particles in a reasonable time frame.

In certain embodiments and further to paragraphs [0004]-[0007] above, ALD coated particles containing one or more therapeutic agent can include biological and/or chemical therapeutics. In some embodiment, the biological and/or chemical therapeutic can include, but are not limited to, polypeptides, proteins, antibody or biologically-relevant fragments thereof, polynucleotides (e.g., RNA, DNA, mRNA, siRNA or the like), oligonucleotides or the like, chimeric molecules, live, attenuated viruses, virus-like particles, lipopolysaccharides, toxins, small molecules, or other suitable therapeutic agent. In some embodiments, coated particles contain at least one anti-microbial agent (e.g., of use as a vaccine against at least one microbial agent). It is contemplated herein that any agent coated by ALD applied coatings can be assessed using compositions and methods disclosed herein for projecting or predicting dissolution rates in order to optimize coating material, coating numbers, coating thickness, or other coating properties for optimal coating of the agent and for optimum delivery of the agent to a subject to treat, ameliorate, and/or prevent at least one health condition.

In some embodiments and further to paragraphs [0004]-[0008] above, at least one therapeutic agent can include one or more polynucleotide encoding at least one antigen. In accordance with these embodiments, the at least one antigen can include, but is not limited to, at least one viral antigen, bacterial antigen, fungal antigen, protozoan-derived agent, toxin, fragment thereof, or a combination thereof. In some embodiments, at least one polynucleotide construct or complex thereof or modified polynucleotide construct or complex thereof can further include another agent including, but not limited to, at least one polypeptide, two or more different polynucleotides, a capsomere, one or more lipids, one or more lipid nanoparticles (LNPs), or other polynucleotide complexable agents or the like.

Disclosed herein and further to paragraphs [0004]-[0009] above, are adaptable, accessible in vitro dissolution compositions and methods which can analyze ALD coating quality, is sufficient to discriminate the dissolution behavior of coated particles of different cycle or coating application numbers and different coat compositions for understanding in vivo dissolution behavior for improved preparation and use of targeted therapeutic agents. In certain embodiments and further to the paragraphs above, dissolution compositions for assessing dissolution rates can include: ALD coated particles and a composition, an aqueous solution, or buffer including, but not limited to, distilled water, phosphate, tris, citrate, nitrate, histidine or other amino acid, a HEPES, an MES, a MOPS, a MOBS, a PIPES, a silicic or other suitable buffer at a pH of about 4.0 to about 10.0 or about 5.0 to about 9.0, or about 5.5 to about 8.0 or about 5.5 to about 7.0, or about to 5.5 to about 6.9, or about 5.8 to about 6.7; or about 5.8 to about 6.5; or about 6.0 to about 6.5, or about 6.1 to about 6.4. In certain embodiments, dissolution compositions for assessing dissolution rates contemplated herein can include a potassium phosphate buffer at a pH of about 5.9 to about pH 6.8, or about pH 6.0 to about pH 6.5. In some embodiments, dissolution compositions for assessing dissolution rates can include ALD coated therapeutic agent-containing particles and a phosphate buffer at a pH of about 6.0 to about 7.0 at a temperature of about 0 °C to about 70 °C, or about 25 °C to about 60 °C, or about 35 °C to about 50 °C.

In some embodiments and further to paragraphs [0004]-[0010] above, the dissolution compositions of use herein can be intermittently or continuously agitated to maintain homogeneity, as needed. In certain embodiments, spectroscopic detection with or without derivatization of a sample, chromatographic separation and detection (e.g., reverse phase, size exclusion, ion exchange etc and MALS, evaporative light scattering, charged aerosol, pulsed amperometric detection, mass spectroscopy, etc.), refractive index, Raman, electrophoresis (capillary, gel, etc.), SPR, ELISA, PCR, particle sizing/imaging/microscopy can be used to detect dissolution of ALD coated therapeutic agent containing particles disclosed herein. In certain embodiments, a time frame for the dissolution can be from about one day to about one week depending on for example, on coat number of a particular ALD coating material being tested. In certain embodiments, the therapeutic agent of an ALD-coated therapeutic agent containing particle can be released in about 30, or about 25, or about 20, or about 15, or about 10 hours or less in a buffer disclosed herein. In certain embodiments, samples can be taken for dissolution analysis at a pre-determined interval (e.g., every 5 minutes to about every 3 hours, if needed) for dissolutions run at higher temperatures (e.g., greater than 25 °C) and higher buffer concentrations (e.g., higher phosphate concentrations) to achieve specificity and projected time of complete dissolution and release. In some embodiments, samples of ALD-coated therapeutic agent containing microparticles in a dissolution buffer disclosed herein can be taken for dissolution analysis about every hour.

In some embodiments and further to paragraphs [0004]-[0011] above, methods for using dissolution compositions and assessing dissolution rates of ALD-coated target therapeutic-agent-containing particles can include suspending ALD-coated target therapeutic-agent-containing particles in a phosphate buffer at a pH of about 5.8 to about 7.0 or about to 6.0 to about 6.7 or about 6.0 to about 6.5 to make a suspension. In other embodiments, heating the suspension, from about 35 °C to about 70 °C (e.g., about 50 °C), to accelerate release of the target therapeutic agent, reduce experimental duration and assess inflection points of release of a therapeutic agent contemplated herein. Optionally, mixing the sample continuously to promote homogeneity of the dissolution composition containing the ALD-coated target therapeutic-agent-containing particles. Incubating the suspension and separating the ALD-coated target therapeutic-agent-containing particles from the dissolution buffer at pre-determined time points and removing a sample for analysis from a remaining supernatant. Optionally, replacing suspension volume and resuspending separated ALD-coated target therapeutic-agent-containing particles, and continuing incubation under selected dissolution conditions and repeating this process to analyze multiple samples over a pre-determined time. Analyzing each sample removed at a time point for release of the target therapeutic-agent from the ALD coated particles.

In certain embodiments and further to paragraphs [0004]-[0012] above, compositions for and dissolution rates of ALD coated particles can be determined. In accordance with these embodiments, these compositions and methods reduce analysis times while also sparing amounts or concentrations of target therapeutic agent needed for these assessments, providing more rapid analysis at reduced cost of time and money compared to standard dissolution methods.

In some embodiments and further to paragraphs [0004]-[0013] above, the at least one therapeutic agent of the ALD-coated therapeutic-agent can include one or more polynucleotide encoding at least one antigen. In accordance with these embodiments, the at least one antigen can include, but is not limited to, at least one viral antigen, bacterial antigen, fungal antigen, protozoan-derived agent, toxin, fragment thereof, or a combination thereof. In some embodiments, at least one polynucleotide construct or complex thereof or modified polynucleotide construct or complex thereof can further include another agent including, but not limited to, at least one polypeptide, two or more different polynucleotides, a capsomere, one or more lipids, one or more lipid nanoparticles (LNPs), or other polynucleotide complexable agents or the like.

Yet other embodiments and further to paragraphs [0004]-[0014] above, disclose kits for assessing dissolution rates of one or more ALD-coated target therapeutic-agent-containing particles disclosed herein, and at least one container.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1B represent exemplary dissolution curves of coated particles after 100 ALD cycles in a buffer solution (FIG. 1A) and the number of hours at the inflection point (C-value from four parameter logistic regression) vs temperature (FIG. 1B), according to various aspects of the disclosure.

FIGS. 2A-2C represent exemplary dissolution curves of coated particles after 100 ALD cycles in buffer solutions having different concentrations (FIG. 2A) or pH (FIG. 2B-2C) according to various aspects of the disclosure.

FIGS. 3A-3B represents exemplary dissolution curves (FIG. 3A) or hours to an inflection point (FIG. 3B) for different ALD coated particles having different coat numbers ranging from 50 to 1000 ALD cycles in a buffer solution according to various aspects of the disclosure.

FIG. 3C represents an exemplary plot of weeks to peak antibody titer in vivo compared to hours at inflection point determined in FIG. 3B for ALD coated particles having different coat numbers (in parenthesis), according to various aspects of the disclosure.

FIG. 3D represents an exemplary model plot showing modeled release of alumina-titania laminates in accelerated dissolution model relative to an alumina only coating, according to a various aspects of the disclosure.

FIGS. 4A-4C represent exemplary dissolution curves for damaged ALD coated particles (FIG. 4A), ALD coated particles having acceptable coat quality (FIG. 4B) or ALD coated particles having unacceptable coat quality (FIG. 4C), according to various aspects of the disclosure.

FIG. 5 represents an exemplary dissolution curve of coated particles after 100 ALD cycles in a buffer solution according to various aspects of the disclosure.

FIGS. 6A-6B represent exemplary dissolution curves of powders from independent lots made using a standard in-house ALD coating method (FIG. 6A), and three independent powders coated using an altered ALD method to intentionally generate poorer coatings (FIG. 6B) according to various aspects of the disclosure.

FIGS. 7A-7B represent exemplary dissolution curves under conditions of either various pH conditions (7A) or buffer conditions (7B) according to various embodiments disclosed herein.

FIG. 8 represents an exemplary model plot demonstrating modeled release of alumina only, alumina-titania, titania only, and alumina-zinc laminates in accelerated dissolution model using a buffer relative to an alumina only coating, according to a various aspects of the disclosure.

FIGS. 9-12 represent exemplary dissolution curves of various coated therapeutic agent containing microparticles with different therapeutic agents and a buffer disclosed herein, according to a various aspects of the disclosure. FIG. 9 illustrates a 100-coat therapeutic agent-containing microparticle with a small molecule therapeutic, according to a various aspects of the disclosure. FIG. 10 illustrates a 130-coat therapeutic agent-containing microparticle with a glycoconjugate therapeutic representative, according to a various aspects of the disclosure. FIG. 11 illustrates a 50-coat therapeutic agent-containing microparticle with a lipid nanoparticle therapeutic (e.g., mRNA-LNP), according to a various aspects of the disclosure. FIG. 12 illustrates a 50-coat therapeutic agent-containing microparticle with an oligonucleotide/polynucleotide therapeutic representative.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some embodiments, well known methods or components have not been included in the description.

An in vitro dissolution assay can be used to understand the mechanisms and kinetics of release in vivo of a targeted therapeutic agent coated by ALD coating or other metallo-organic, metal oxide, or metal alkoxide coated therapeutic agent-containing microparticle. These in vitro methods have utility from at least a quality control perspective. However, applying an in vitro dissolution assay for these purposes is difficult when a coated targeted therapeutic agent is designed to be poorly soluble, inaccessible, and/or designed to enable delayed release of the targeted therapeutic agent in a clinical setting. (e.g., when administered to a subject). These issues are relevant to ALD coated therapeutic agent containing microparticles. In some embodiments, these issues are relevant to ALD-coated therapeutic agents designed for use as single shot, delayed release therapeutic agents (e.g., an immunogenic agent such as vaccines or anti-cancer agent). These single shot compositions can include the same or different therapeutic agents in the same or different coating layers of ALD coated particles. Generation of these coated particles involves applying at least one metal-ion or metallo-organic coating layer (e.g., alumina, silica, zinc or the like) of a pre-determined thickness to spray dried, lyophilized and/or thermostabilized therapeutic agent-containing particles, microparticles or nanoparticles. In accordance with these embodiments, this layering occurs using a pre-determined number of ALD cycles to coat the thermal stable therapeutic agent-containing particle to enable release of the therapeutic agent contents at a specified time, or times, post-injection or administration. Because the time-to-release can span from about 1 hour, or up to a few hours, or up to days, weeks, or months from the time of delivery, it is challenging to create a model for analyzing dissolution of these coated particles (e.g., ALTA® particles) in a reasonable time frame at a reduced cost in time, material and money. Embodiments disclosed herein provide for adaptable and accessible in vitro dissolution methods able to interrogate ALD coat quality, discriminate the dissolution behavior of ALD-coated products (e.g., ALTA™) of different cycle numbers and of different coat compositions for understanding in vivo dissolution behavior and/or parameters of any ALD-coated therapeutic agent. In accordance with these embodiments, in vivo dissolution behavior and/or parameters can include one or more of uniformity of dispersion of a therapeutic agent from the coating, integrity of coating layers, optimal coating layers, predictability of dissolution timing of one or more therapeutic agent doses of the same or different therapeutic agents at the same or different times of dissolution.

Embodiments of the present disclosure provide novel compositions for use in analyzing dissolution rates of ALD coated therapeutic agent-containing particles (or microparticles or nanoparticles) and methods for determining ALD coating quality and predictability of dissolution, for example. In certain embodiments, in vitro methods can be used to determine in vivo dissolution rates of ALD coated therapeutic agent-containing particles, microparticles and/or nanoparticles. In some embodiments, formulations or compositions disclosed herein include agents for optimal analysis of dissolution rates of insoluble or highly insoluble ALD coated therapeutic agent-containing particles. In accordance with these embodiments, compositions disclosed herein can be used to provide improved conditions for determining ALD coating quality and/or dissolution rates. In some embodiments, improved coating compositions, coating cycles, coating layering and thickness can be determined for improved delivery and administration of pharmaceutical products with improved outcomes, predictability of timing of exposure to a target subject, and reliable concentration delivery of the therapeutic agent to the subject. Dissolution compositions and methods disclosed herein enable consistent, specific, relatively rapid assessment of coat quality and dissolution behavior of encased material such as therapeutic agents which in an in vivo setting can typically take upwards of months to release a therapeutic agent from its ALD coatings by standard methods. In certain embodiments, compositions and dissolution methods disclosed herein can be used as quality control assays for ALD-coated particles, for specific and consistent assessment of ALD-coated therapeutic agent-containing particles of same coat number, can discriminate between different coat numbers of ALD-coated particles and can quickly detect damaged/leaky ALD-coated therapeutic agent-containing particles contemplated herein.

It is known in the art that dissolution methods are typically performed at 37 °C to mimic or represent in vivo conditions and can require substantial quantities of product and can also require sophisticated dissolution apparati for conducting such analysis at considerable costs of time and money. These standard dissolution methods are typically limiting in cases where a dissolution apparatus is unavailable, a therapeutic agent is limited, and the use of physiological temperatures results in excessive experimental run times often requiring precautions to mitigate microbial interference (e.g., microbial contamination) as well as other issues. Embodiments of the instant inventions provide advancements to assessing dissolution rates of ALD coated therapeutic agent containing particles compared to those known in the art. For example, methods disclosed herein do not require mimicking of physiologically relevant conditions to achieve an in vitro-in vivo (IV/IV) correlation and predict or project dissolution rates and parameters of ALD coated therapeutic agent containing particles which provides, for example, a more reliable, reproducible assay in a reduced period of time for analysis. Compositions and methods disclosed herein do not require filtering or dialysis to provide relevant information regarding dissolution rates and time of inflection and other relevant dissolution-related information. Filtering and dialysis can be optional. Compositions and methods disclosed herein are an advantage over those assays known in the art that can provide inaccurate or unreliable results due to adsorption/loss of target, clogging (e.g., a filter), rate limiting diffusion through a membrane or the like. Further, certain embodiments disclosed herein for assessing dissolution rates of coated therapeutic agent containing particles do not require mechanical disruption used in compendial dissolution methods (e.g., using a paddle to stir or a piston or other related device to disrupt clumping during a standard dissolution assay), which would likely disrupt ALD coatings and likely inaccurately model in vivo dissolution behavior. In certain embodiments, gentle agitation of particle containing compositions can be used instead of mechanical disruption. One other advantage of dissolution compositions and methods disclosed herein compared to standard methods is that volume of the assayed material is flexible and adaptable to conform to the particles being assayed.

In some embodiments and further to paragraphs [0029]-[0031] above, methods for performing dissolution analysis on samples disclosed herein can be performed in simple, low protein-binding tubes, multi-well plates, microtiter plates, or dishes in a temperature-controlled environment using 1.0 gram or less, 750 mgs or less, 500 mgs or less, 250 mgs or less, 150 mgs or less, or about 100 mgs or less of ALD-coated therapeutic agent-containing particles (e.g., less than 100 mg of essentially dry coated particles). In other embodiments, while duration of the dissolution experiment can depend on composition and/or number of ALD coating layers of therapeutic-agent-containing particles, as disclosed herein, full release of metallo-organic or metal coating layers (e.g., alumina) material disclosed herein is within one month or less, within three weeks or less, within two weeks or less, within one week or less, within 5 days or less, within 3 days or less, within a day or less. In some embodiments, particles disclosed herein can be coated with a high number of coating layers from about 10 layers up to about 1000 or more, or about 10 layers up to about 750, or about 10 layers up to about 500, or about 10 layers up to about 250 or about 50 layers up to about 150, or more coating layers. In some embodiments, particles disclosed herein can be coated with 5 layers up to about 40 coating layers to obtain complete enclosure of therapeutic-agent-containing particles.

In other embodiments and further to paragraphs [0029]-[0032] above, as disclosed herein parameters or conditions of dissolution assays such as temperature, buffer concentration, and pH can be further adapted based on coat composition and number of layers of ALD coated therapeutic agent-containing particles contemplated herein. In accordance with these embodiments, detection of release of any therapeutic agent contemplated herein of these coated microparticles is neutral or generally applicable to a target therapeutic agent (e.g., the active pharmaceutical ingredient (API)) as well as the coating composition applied under ALD conditions. As disclosed herein representative therapeutic agents (e.g., use of ovalbumin in an exemplary method) is representative of a therapeutic agent of ALD coated therapeutic agent-containing particles contemplated herein. In certain embodiments, methods for detection of one or more released therapeutic agent can be performed using chromatographic, fluorescence, or absorbance methods or other methods known in the art to detect a target or desired therapeutic agent. In some embodiments, using chromatographic methods disclosed herein and/or fluorescent derivatization with o-phthalaldehyde (OPA) for example, can detect premature release of one or more therapeutic-agents and/or API from damaged or defectively coated particles. In other embodiments, time to an inflection point (e.g., at least about 40%, at least about 50%, at least about 60%, at least about 70%, at least about 80%, at least about 90%, essentially full or complete therapeutic agent release from ALD coated particles) can be reproducibly modeled within about 1 day, within about 18 hours, within about 15 hours, within about 12 hours, within about 9 hours, within about 6 hours, within about 4 hours, or within about a 2-hour window of time, for therapeutic agent of the same ALD coat number of the therapeutic agent-containing particles and of the same therapeutic agent making these dissolution assays useful as a quality control assay for understanding and creating improved and consistently produced ALD-coated therapeutic agent containing particles (e.g., ALTA™ products).

In some embodiments and further to paragraphs [0029]-[0033] above, in vitro dissolution assays can be used to understand mechanisms and kinetics of release in vivo of a targeted ALD-coated therapeutic agent or therapeutic agents. In other embodiments, in vitro dissolution assays can be used to understand mechanisms and kinetics of release in vivo of a targeted ALD-coated therapeutic agent containing microparticles for quality control and improved accuracy of timing of delivery and dosing. In certain embodiments, in vitro dissolution assays disclosed herein are designed to assess dissolution of poorly soluble delayed release therapeutics. In some embodiments, ALD coated therapeutic-agent containing particles designed as single shot, delayed release therapeutic agents' dissolution rates can be assessed by compositions and methods disclosed herein. In some embodiments, ALD coated thermostable therapeutic agent-containing particles' dissolution rates can be assessed by compositions and methods disclosed herein. In certain embodiments, in vitro dissolution compositions and assays disclosed herein can assess an inflection point of dissolution of poorly soluble delayed release therapeutics agents of ALD coated thermostable therapeutic agent-containing particles contemplated herein. In accordance with these embodiments, inflection points of targeted ALD coated thermostable therapeutic agent containing particles to release the thermostable therapeutic agent(s) or to release the thermostable therapeutic agents contained within the ALD coated thermostable therapeutic agent-containing particles. Further, as demonstrated herein, a positive correlation was identified between hours to an inflection point of therapeutic agent(s) of ALD coated thermostable therapeutic agent-containing particles released in vitro and for example, with respect to an immunogenic agent, time to maximum antibody titer detected in vivo of therapeutic or immunogenic agents contained withing the ALD coated particles.

In some embodiments and further to paragraphs [0029]-[0034] above, ALD coated therapeutic-agent containing particles disclosed herein can include ALD applied metal oxide or metallo-organic material coated particles containing at least one thermostable antigen or therapeutic agent with a defined number of ALD coatings to enable release of the at least one thermostable antigen or therapeutic agent at a specified time, or times, post-administration of the coated microparticles. In certain embodiments, the metal oxide coating layers can include aluminum oxide, an aluminum alkoxide (e.g., alucone), silicon dioxide (SiO₂), titanium dioxide (TiO₂), zinc oxide (ZnO₂), zirconium oxide (ZrO₂), or silicon nitride (Si₃N₄) alone, in alternating layers, or other suitable layer pattern, or in a suitable combination composition for layering on the at least one thermostable antigen or therapeutic agent-containing particles contemplated herein. In some embodiments, the metal oxide coating layers can include aluminum oxide alone, in alternating layers, or in a combination layer composition.

In certain embodiments and further to paragraphs [0029]-[0035] above, the ALD applied coating layers to the at least one therapeutic-agent containing microparticle, at least one thermostable antigen or the at least one therapeutic agent- or thermostable antigen-containing particles contemplated herein can be about 0.1 nm to about 40.0 nm in thickness, 0.1 nm to about 30.0 nm in thickness about 0.1 nm to about 20 nm in thickness, 0.1 nm to about 10.0 nm in thickness, or about 0.1 nm to about 7.5 nm in thickness each or in part or in total. In some embodiments, the at least one thermostable antigen or the at least one therapeutic agent-containing particles can include coating layers sufficient to uniformly coat the at least one thermostable antigen or therapeutic agent-containing particles to delay release or provide a timed-release of the at least one thermostable antigen or therapeutic agent from ALD coated particles from the central or innermost therapeutic agent, thermostable antigen, or immunogenic agent-containing particle. In other embodiments, coatings of desired thickness relative to the at least one thermostable antigen or therapeutic agent-containing particles or thermostable particles (e.g., spray dried particles) can include a defined number or defined range of ALD cycles to add for enabling or permitting release of the at least one therapeutic agent, thermostable antigen, or immunogenic agent at a specified time, or at specific times, post-administration to a subject, such as within minutes, within hours, within a day, within a week, within 2 weeks or more, within a month or within 2 or more months after administration or delivery of a formulation disclosed herein to a subject. It is understood by one of skill in the art that number of ALD-applied coatings to therapeutic agent-containing particles dictates the timing of release upon administration and compositions and methods disclosed herein can be used to fine-tune coating layer number to timing of release of a target therapeutic agent contemplated herein.

In certain embodiments and further to paragraphs [0029]-[0036] above, total ALD-applied coats of the at least one thermostable antigen or therapeutic agent-containing particles or thermostable particles can be from about 5 coats to more than 1,500 coats, or about 10 coats to about 1,000 coats, or about 20 coats to about 1,000 coats, or about 30 coats to about 800 coats, or about 30 coats to about 400 coats, or about 30 coats to about 300 coats or at least 30 to at least 50 coats. In accordance with these embodiments, the number of ALD-applied coats to the at least one thermostable antigen or therapeutic agent-containing particles or thermostable particles will depend on the intended date of exposure post administration to a subject, for example, a few hours, to a day, to several days, to a week, to a couple to several weeks, to a month, to a couple of months, to several months, to 6 months, to 9 months, to a year or more or any time period in between. In accordance with these embodiments, dissolution assays disclosed herein can be used to analyze and determine ideal number of coating layers for timed release of a targeted therapeutic or for targeted therapeutics contemplated herein. In certain embodiments, a lower number of coating layers (e.g., less than 250) for a targeted therapeutic can be used when the desired dissolution timing of the coated particles is about 3 weeks or less. In certain embodiments, assay timing for naturally-occurring or real-time dissolution rates of coated particles disclosed herein in vitro as representative of in vivo can be within hours to within 3 weeks or more or any time in between. In accordance with these embodiments, any ALD coated therapeutic agent-containing particle contemplated herein can undergo an accelerated dissolution rate compared to real-time and in certain embodiments disclosed herein, up to 5 times, up to 10 times, up to 15 times, up to 20 times, up to 25 times, up to 50 times, up to 75 times, or up to 100 times faster or more, compared to current methods known in the art and compared to real-time dissolution of coating layers of a target ALD coated particle. In certain embodiments, ALD coated therapeutic-agent containing particles for dissolution at 1 month, 2 months, 3 months, 4 months, 5 months, 6 months or more or in between can be analyzed by compositions and methods disclosed herein within hours, within a day, within a few days or more, up to about 3 weeks compared to real-time dissolution of the ALD coated therapeutic agent containing particles. In some embodiments, further analysis of dissolution of ALD coated therapeutic agent containing particles can predict or project an inflection peak or inflection peaks as appropriate, of the ALD coated therapeutic agent containing particles and/or antibody expression thereof, if relevant (e.g., when the therapeutic agent is an immunogenic agent (e.g., a vaccine). In accordance with these embodiments, prediction or projection of inflection peak of any coated therapeutic agent or therapeutic agents contemplated herein can provide increased accuracy for projections of treatments or preventions of a targeted health condition (e.g., infection by a pathogen, treatment of cancer or chronic health condition, etc.) with respect to a target therapeutic agent.

In certain embodiments and further to paragraphs [0029]-[0037] above, coated particles containing one or more therapeutic agent can include any biological or chemical or other therapeutic agent capable of being thermostabilized. In accordance with these embodiments, any therapeutic-agent of any ALD coated therapeutic-agent containing particle disclosed herein is considered inert or independent of, dissolution methods disclosed. For example, an ALD coated therapeutic-agent containing particle contemplated herein can contain a therapeutic agent which can be in an essentially dry form (e.g., spray-dried into a glassy matrix) coated or surrounded by metallo-organic, metal-containing, or other ALD applied coating layer(s). In other embodiments, number of coating layers of a therapeutic-agent containing particle can be decided based on desired timing of release (or desired timing of antibody expression, if appropriate) of the therapeutic-agent or therapeutic agents (e.g., multiple doses of the same therapeutic agent or different therapeutic agent coated within a single particle in the same or different layers). In certain embodiments, neutralization assays, detection assays, or the like can be used to assess timing of an immune response or assay in reference to an inflection peak, for example, of dissolved or dispersed therapeutic-agent of therapeutic-agent containing ALD coated particles contemplated herein. In some embodiments, the biological or chemical therapeutic or therapeutic or immunogenic agent disclosed herein can include, but are not limited to, polypeptides, proteins, polynucleotides (e.g., mRNA, cDNA, DNA, RNA, siRNA etc.), oligonucleotides, chimeras, or polynucleotide construct or modified polynucleotide construct or complex thereof, live, attenuated viruses, live virus, virus-like particles, an inactivated virus, a toxoid, toxins, bacteria, bacterial antigen, bacterial derived agent, lipopolysaccharides, small molecules, antibodies, monoclonal antibodies, segments thereof, fragments thereof, recombinants thereof or other suitable therapeutic agent. In some embodiments, the coated particles contain at least one anti-microbial agent or immunogenic agent (e.g., of use as a vaccine). In certain embodiments, thermostable therapeutic agents disclosed herein can include, but are not limited to, an anti-microbial that includes, but is not limited to, an anti-viral, anti-bacterial, anti-fungal, anti-protozoan thermostable therapeutic agent.

In some embodiments and further to paragraphs [0029]-[0038] above, at least one therapeutic agent can include one or more polynucleotide encoding at least one antigen. In accordance with these embodiments, the at least one antigen can include, but is not limited to, at least one viral antigen, bacterial antigen, fungal antigen, protozoan-derived agent, toxin, fragment thereof, or a combination thereof. In some embodiments, at least one polynucleotide construct or complex thereof or modified polynucleotide construct or complex thereof can further include another agent including, but not limited to, at least one polypeptide, two or more different polynucleotides, a capsomere, one or more lipids, one or more lipid nanoparticles (LNPs), or other polynucleotide complexable agents or the like.

In some embodiments and further to paragraphs [0029]-[0039] above, disclosed herein are adaptable, accessible in vitro dissolution compositions and methods which can analyze ALD coating quality, is sufficient to discriminate the dissolution behavior of coated particles of different cycle or coating application numbers and different coat compositions for understanding in vivo dissolution behavior for improved preparation and use of targeted therapeutic agents. In certain embodiments and further to the paragraphs above, dissolution compositions for assessing dissolution rates can include ALD coated particles and at least one of: distilled water, a phosphate, a citrate, a tris, a nitrate, a borate, a sulfate, a histidine, a HEPES, a MES, a MOPS, a MOBS, a PIPES, a silicic or other suitable buffer or a combination thereof. In accordance with these embodiments, buffers for dissolution of coated microparticles disclosed herein can be at a pH of about 4.0 to about 10.0, or about pH 5.0 to about pH 9.5, or about pH 5.0 to about pH 9.0, or about pH 5.0 to about pH 8.5, or about pH 5.0 to about pH 8.0, or about pH 5.0 to about pH 7.5, or about pH 5.0 to about pH 7.0, or about pH 5.5 to about pH 7.0, or about pH 5.5 to about pH 6.5, or about pH 5.8 to about pH 6.5 or about pH 5.8 to about pH 6.3. In some embodiments, pH of a buffer can depend on the buffer selected for a particular coating composition (e.g., a buffer for alumina coated or alumina and titanium coated therapeutic-agent-containing particles). In certain embodiments, a phosphate buffer for alumina coated or alumina and titanium coated therapeutic-agent-containing particles can be used for efficient dissolution assay analysis. In some embodiments, dissolution compositions for assessing dissolution rates can include ALD coated particles and a phosphate buffer at a pH of about 5.8 to about 6.8 at a temperature of about 20 °C to about 70 °C, or about 25 °C to about 60 °C, or about 30 °C to about 50 °C, or about 35 °C to about 45 °C. In certain embodiments, the buffer can be a potassium salt buffer. In accordance with these embodiments, the potassium buffer can include a potassium phosphate or other potassium salt buffer and optionally, further include a second salt (e.g. sodium chloride) In some embodiments, dissolution compositions for assessing dissolution rates can include ALD coated particles and a phosphate buffer at a pH of about 5.0 to about 8.0, or about 6.0 to about 7.0 at a temperature of about 20 °C to about 60 °C, 35 °C to about 70 °C, or about 40 °C to about 70 °C, or about 40 °C to about 65 °C, or about 40 °C to about 55 °C, or 45 °C to about 55 °C, or about 50 °C to about 60 °C, or about 50 °C or any temperature in between. In some embodiments, the dissolution compositions including a composition (e.g. introduced powder or particles) of ALD-coated therapeutic agent-containing microparticles can be intermittently or continuously agitated, mixed or vibrated by any method known in the art, to maintain homogeneity, as needed. In certain embodiments, phosphate buffers disclosed herein can include a potassium phosphate buffer and/or a sodium phosphate buffer. In certain embodiments, buffers disclosed herein do not include a sulfate buffer. In accordance with these embodiments, buffers disclosed herein do not use a sulfate buffer (e.g. sodium sulfate) when 60% or more, 70% or more, 80% or more, 90% or more or up to 100% therapeutic agent release is desired. In other embodiments, the buffer can include a citrate buffer for example, as a combination buffer with another buffer having slower dissolution rates. In certain embodiments, the buffer does not include a citrate buffer. In some embodiments, the buffer does not include a sodium citrate buffer. In some embodiments, buffers can further include a salt component such as sodium, potassium, magnesium, calcium, copper, or the like. In certain embodiments, the salt is potassium.

In some embodiments and further to paragraphs [0029]-[0040] above, buffers of use herein can further include a chelating agent. In certain embodiments, a chelating agent can be used to accelerate dissolution of a more difficult to dissolve ALD coating composition. In accordance with these embodiments, a chelating agent can be included to facilitate recovery of a released therapeutic agent or agents. In accordance with these embodiments, a chelating agent can include, but is not limited to, EDTA, EDPA, TTHA (Triethylenetetramine-N,N,N′,N″,N′″,N′″-hexaacetic acid), ascorbate, histidine, 4-Hydroxy-5-methyl-3-pyridinecarboxylic acid, deferoxamine, Feralex-G, Desferal, Deferiprone and derivatives thereof, fluoride, maltol, citrate and derivatives thereof, hydroxyurea, dihydroxyacetone, acetylacetone, salicylate, lactic acid, Triapine (3-aminopyridine-2-carboxaldehyde thiosemicarbazone), Nitrilotri(methylphosphonic acid), nitriloacetic acid, cacodylate, succimer (DMSA), diethylenetriaminepentaacetate (DTPA), N,N,N′,N′-tetrakis(2-pyridylmethyl)-ethylenediamine (TPEN), bicarbonate, catechol, and any derivative thereof or the like. In certain embodiments, the buffer comprises a chelating agent in a potassium phosphate buffer. In some embodiments, the buffer comprises a chelating agent in a potassium phosphate buffer at a pH of about 5.5 to about 7.5, about 5.5 to about 7.0, about 5.5 to about 6.5, or about 5.8 to about pH 6.4, or about pH 6.0 to about pH 6.3. In certain embodiments, the buffer does not include sodium phosphate. In accordance with these embodiments, inclusion of a sodium phosphate buffer instead of a potassium phosphate buffer for dissolution methods disclosed herein can accelerate dissolution of the therapeutic-agent containing ALD coated particles compared to potassium phosphate buffer alone, if more rapid dissolutions are sought or for predicting or scheduling dissolution timing of targeted therapeutic agent; for example, identifying coating materials and number of coating layers desired for a certain projected dissolution outcome in vivo. In some embodiments, a sodium phosphate buffer can be combined with a potassium phosphate buffer for use in dissolution methods disclosed herein to accelerate dissolution of the therapeutic-agent containing ALD coated particles if more rapid dissolutions are sought or for predicting or scheduling dissolution timing of targeted therapeutic agent; for example, identifying coating materials and number of coating layers desired for a certain projected dissolution outcome in vivo. In some embodiments, sodium phosphate buffers disclosed herein can have a pH of about 5.0 to about 8.0 or about pH 5.5 to about pH 7.5 or about pH 5.5 to about 6.5 or about pH 6.0.

In certain embodiments and further to paragraphs [0029]-[0041] above, buffers for use in dissolution assays disclosed herein include, but are not limited to, phosphate, tromethamine (Tris), or other buffer having similar properties as phosphate and/or tris buffer and/or capable of maintaining a pre-selected pH (e.g., pH 4.0-10.0 or about 5.0 to about 9.0, or about 5.5 to about 8.5, or about 5.5 to about 8.0, or about 5.5 to about 7.5, or about 5.5 to about 7.0 or about 5.8 to about 6.5 or about 6.0 to about 6.3). In some embodiments, dissolution compositions for assessing dissolution rates of ALD coated therapeutic agent-containing particles disclosed herein can include the ALD coated particles and a phosphate and/or tris buffer at a pH of about 5.0 to about 8.0 at a temperature of about 25 °C to about 70 °C. In certain embodiments, dissolution compositions for assessing dissolution rates disclosed herein can include ALD coated particles and a buffer (e.g., phosphate or tris buffer) at a concentration of greater than 10.0 mM up to about 1.0 M or about 20 mM to about 500 mM or about 30 mM to about 300 mM, or about 40 mM to about 300 mM, or about 50 mM to about 300 mM, about 100 mM to about 300 mM at a pH of about 5.0 to about 8.0 at a temperature of about 35 °C to about 70 °C. In some embodiments, a slight decrease or increase in buffer mM concentration from about 70 mM up to about 350 mM can be used when fine-tuning the assay to dissolve a target coating layer slower or faster respectively, depending on desired outcome. In accordance with these embodiments and those disclosed herein, a phosphate, tris, or other disclosed or contemplated buffer concentration can be about 1.0 mM to about 1.5 M, or about 10.0 mM to about 1.0 M, to about 50.0 mM to about 500.0 mM, or about 50.0 mM to about 250.0 mM, or about 50.0 mM to about 200.0 mM, or about 50.0 mM to about 150.0 mM, or about 100.0 mM or any concentration in between. In certain embodiments, a phosphate, tris, or other buffer concentration can be about 100.0 mM.

In other embodiments and further to paragraphs [0029]-[0042] above, dissolution compositions for assessing dissolution rates can include ALD coated therapeutic agent-containing particles disclosed herein and a buffer (e.g., phosphate, citrate or tris buffer) at a pH of about 5.0 to about 8.0, or about pH 5.5 to about pH 7.5, or about pH 6.0 to about 7.0, or about pH 6.5. In some embodiments, dissolution compositions for assessing dissolution rates of therapeutic agents of ALD coated therapeutic agent-containing particles disclosed herein can include about 0.1 mg/ml to about 1.0 g/ml, about 0.5 mg/ml to about 500.0 mg/ml, about 1.0 mg/ml to about 250.0 mg/ml, about 5.0 mg/ml to about 150.0 mg/ml, about 5.0 mg/ml to about 50.0 mg/ml, about 5.0 mg/ml to about 25.0 mg/mg, about 10 mg/ml or any concentration in between of ALD coated therapeutic-agent containing particles and a buffer at a pH of about 5.5 to about 7.0 or about 5.8 to about 6.5 at a temperature of about 35 °C to about 70 °C. In some embodiments, dissolution compositions for assessing dissolution rates can include about 0.1 mg/ml to about 1.0 gm/ml, about 0.5 mg/ml to about 500.0 mg/ml, about 1.0 mg/ml to about 250.0 mg/ml, about 5.0 mg/ml to about 150.0 mg/ml, about 5.0 mg/ml to about 50.0 mg/ml, about 5.0 mg/ml to about 25.0 mg/mg, about 10 mg/ml ALD coated therapeutic agent-containing particles disclosed herein and a buffer at a pH of about 5.5 to about 7.0 at a temperature of about 35 °C to about 70 °C, or about 40 °C to about 70 °C, or about 45 °C to about 70 °C, or about 45 °C to about 65 °C, or about 50 °C to about 60 °C, or about 50 °C or any temperature in between. In some embodiments, ALD coated therapeutic agent-containing particles in dissolution compositions can be intermittently or continuously agitated, mixed or stirred to maintain homogeneity, as needed. In accordance with these embodiments, essentially all of the therapeutic agent of ALD coated therapeutic agent-containing particles disclosed herein can be released under conditions disclosed herein for predicting, manufacturing and/or scaling up ALD coated therapeutic agent-containing particles having specific coating materials and specific numbers of coating layers for more accurate and dependable timed-release of a targeted therapeutic agent in vivo contemplated herein.

In some embodiments and further to paragraphs [0029]-[0043] above, methods for using dissolution compositions and assessing dissolution rates of ALD-coated target therapeutic-agent-containing particles are disclosed. In accordance with these embodiments, methods for using dissolution compositions and assessing dissolution rates of ALD-coated target therapeutic-agent-containing particles can include suspending ALD-coated target therapeutic-agent-containing particles in a buffer (e.g., phosphate or tris buffer, other disclosed buffer or the like) at a pH of about 5.0 to about 7.5, or about 5.5 to about 7.0, or about 5.5 to about 6.5, to make a suspension. Heating the suspension, from about 35 °C to about 70 °C, about 35 °C to about 60 °C (e.g., about 50 °C), to accelerate release of the target therapeutic agent and reduce experimental duration to inflection point of release (e.g., essentially full release of a target therapeutic agent). Optionally, mixing or agitating the coated particle-containing dissolution composition intermittently, at pre-determined times, or continuously to promote homogeneity of the dissolution composition containing the ALD-coated target therapeutic-agent-containing particles. Further, incubating the suspension or composition and separating any remaining ALD-coated target therapeutic-agent-containing particles from the dissolution buffer at pre-determined time points and removing a sample for analysis from a remaining supernatant. Optionally, in other embodiments, replacing suspension volume with additional buffer under the same or similar conditions as buffer removed and resuspending separated or remaining ALD-coated target therapeutic-agent-containing particles, and continuing incubation under selected dissolution conditions, removing a sample at, at least at a second time interval, and repeating this process to analyze one, a few or multiple samples over a pre-determined time. In some embodiments, each sample removed can be analyzed at a time point for scheduled release of the target therapeutic-agent of the ALD coated therapeutic agent-containing particles. In accordance with these embodiments, inflection times of a particular therapeutic agent of therapeutic agent-containing coated microparticles can be assessed to optimize coating types and number of layers for correlating a particular outcome in vivo. In addition, as applicable (e.g., for an immunostimulatory agents or vaccines), antibody assays can be performed and correlation of inflection time and maximum antibody expression using a neutralization assay can be assessed or predicted/projected. It is understood by one of skill in the art that there can be a delay of several minutes up to several days between inflection time (e.g., optimum dissolution timing of an agent) and an immune response to a targeted therapeutic agent. These and other effects can be considered when assessing ideal coating conditions of an immunogenic agent, for example. In certain embodiments, therapeutic agent-containing particles can contain more than one dose of the same or different therapeutic agents. Dissolution assays disclosed herein can be used to assess and implement ideal coating layers and coating compositions for a given therapeutic agent-containing particle for optimal dissolution and immune response or therapeutic response in a subject.

In certain embodiments and further to paragraphs [0029]-[0044] above, compositions for and dissolution rates of ALD coated therapeutic agent-containing particles can be determined. In accordance with these embodiments, these compositions and methods can significantly reduce analysis times while sparing the amount of target therapeutic agent needed in these assessments providing more rapid analysis at reduced cost of time and money compared to standard dissolution methods. It is known in the art that real-time dissolution assays can take several days weeks and up to months or more compared to compositions and methods disclosed herein.

Yet other embodiments and further to paragraphs [0029]-[0045] above, kits for assessing dissolution rates of one or more ALD-coated target therapeutic-agent-containing particles are disclosed and can include at least one container. In accordance with these embodiments, kits can include one or more dissolution buffers. In accordance with these embodiments, the one or more dissolution buffers disclosed herein can be at a particular concentration and at a pre-determined pH or pH range. In other embodiments, kits can include a multi-well plate. In other embodiments, kits can include an apparatus for heating the dissolution buffer in the presence of ALD-coated particles to be analyzed. In yet other embodiments, kits can include neutralization assay components, if appropriate. In yet other embodiments, kits can include an agitation device for continuous or intermittent mixing of the compositions.

In some embodiments and further to paragraphs [0029]-[0046] above, ALD coated therapeutic agent-containing particles for analysis herein can include but are not limited to one or more polynucleotide construct or complex thereof or modified polynucleotide construct or complex thereof can include, but is not limited to, a polynucleotide or modified polynucleotide construct or complex thereof. In accordance with these embodiments, the one or more polynucleotide construct or complex thereof or modified polynucleotide construct, conjugated polynucleotide or complex thereof can include, but are not limited to, any form of one or more of DNA, siRNA, RNA (e.g., mRNA, mRNA-lipid nanoparticle (mRNA-LNP), other mRNA delivery vehicle or conjugate, or the like), or a mixture or complex thereof. In certain embodiments, one or more polynucleotide construct or complex thereof or modified polynucleotide construct or complex thereof can include, but is not limited to, a polynucleotide or complex thereof encoding one or more therapeutic agents or wherein the polynucleotide or modified polynucleotide construct or complex thereof is therapeutically relevant in absence of encoding a secondary molecule including, but not limited to, one or more antigens or immunogenic agent; or includes at least one polynucleotide capable of inducing an immune response in a subject or has immunomodulatory properties in a subject. In certain embodiments, one or more polynucleotide construct or complex thereof or modified polynucleotide construct or complex thereof can include, but is not limited to, an immunogenic agent used as a vaccine, further ALD-coated by compositions and methods known in the art.

In certain embodiments and further to paragraphs [0029]-[0047] above, ALD coated therapeutic agent-containing particles can include particles containing one or more polynucleotide construct or complex thereof or modified polynucleotide construct or complex thereof encoding one or more therapeutic agents or wherein the polynucleotide or modified polynucleotide construct or complex thereof is therapeutically relevant in absence of encoding a secondary molecule In accordance with these embodiments, ALD coated therapeutic agent-containing particles can include, but are not limited to, ALD coated therapeutic agent-containing particles containing a single lipid nanoparticle or a mixture of lipid nanoparticles (LNPs) for encapsulating the polynucleotide construct or complex thereof or modified polynucleotide construct or complex thereof encoding one or more therapeutic agents and/or immunogenic agents capable of inducing an immune response to at least one microbial or microbial-derived agent (e.g., pathogenic viral, bacterial, fungal, protozoan, toxin or other pathogenic microbe).

In some embodiments and further to paragraphs [0029]-[0048] above, a pre-dried or pre-spray-dried liquid formulation containing at least one therapeutic agent for ALD coating can contain one or more polynucleotide construct or complex thereof or modified polynucleotide construct or complex thereof further which can further include, but is not limited to, one or more nonionic starch derivative, other starch, polysaccharide, disaccharide, non-reducing disaccharide agent, glassy agent, or substitutable agent thereof. In accordance with these embodiments, the nonionic starch derivative or substitutable agent thereof includes, but is not limited to, one or more of hydroxyethyl starch, succinylated gelatin, and the like or combinations thereof. In some embodiments, at least one nonionic starch or derivative thereof can be present in the aqueous formulation at a weight-to-volume (w/v) concentration from about 0.1% to about 40.0%, from about 0.1% to about 30.0%, from about 0.1% to about 20%, or from about 0.1% to about 15%, or about 12%. In certain embodiments, the non-reducing disaccharide agent can include trehalose, sucrose, glycine and mannitol, and/or glycine and optionally, at least one smoothing agent (e.g. nonionic starch including, but not limited to, hydroxyethyl starch). In some embodiments, a pre-dried or pre-spray dried liquid formulation containing one or more polynucleotide construct or complex thereof or modified polynucleotide construct or complex thereof further includes, but is not limited to, one or more one or more polymers including, but not limited to, polyvinyl alcohol (PVA), polyacrylic acid (PAA), polyethylene glycol (PEG), povidone, polymethacrylate-based copolymers (e.g., Eudragit), other lipid, other medium to high molecular weight polymers, or combinations thereof.

It is contemplated herein that a thermostable therapeutic agent, pharmaceutical agent or antigen capable of being coated by ALD coating methods can contain any agent capable of assisting with or maintaining the thermostable pharmaceutical agent or antigen in an essentially dry, stable state to maintain stability of the therapeutic and/or thermostable pharmaceutical agent or antigen during and after the ALD coating process. In accordance with these embodiments, any and all ALD coated therapeutic agent-containing particles, ALD coated thermostable pharmaceutical agent, or antigen is capable of being analyzed for coat integrity, stability, coat composition, coating layers, and predictability of delivery using dissolution compositions and methods disclosed herein.

In some embodiments and further to paragraphs [0029]-[0050], one or more polypeptide, protein or fragment thereof, antibody or fragment thereof, small molecule or polynucleotide construct or complex thereof or modified polynucleotide construct or complex encoding at least one therapeutic agent thereof is or encodes at least one antigen specific to one or more of any viral or bacterial pathogen including, but not limited to, any influenza virus, human papilloma virus (HPV or any HPV serotype or type), filovirus (e.g., Ebola virus), Banna virus (BAV), togavirus, alphavirus (e.g. chikungunya), orthobunyavirus, norovirus, polyomavirus, vesiculovirus, coltivirus, orthopoxvirus, enterovirus, orthonairovirus, tanzavirus, flavivirus, thogotovirus, cardiovirus, lymphocryptivirus, orthohantavirus, hepatovirus, hapcivirus, deltavirus, pegivirus, mastodenovirus, mamastrovirus, bocaparvovirus, alphacorona virus, cosavirus, herpes virus (e.g., cytomegalovirus), retrovirus, pneumovirus, papilloma virus (e.g. human papilloma virus (HPV), respirovirus, orthorubulavirus, corona virus, polyomavirus, arenavirus, picornavirus or other pathogenic virus. In certain embodiments, one or more polypeptide, protein or fragment thereof, antibody or fragment thereof, small molecule or polynucleotide construct or complex thereof or modified polynucleotide construct or complex encoding at least one therapeutic agent thereof is or encodes at least one antigen specific to one or more of poliovirus, norovirus, rotavirus, hepatitis A, hepatitis, B, hepatitis C, dengue virus (dengue 1-4 or other), Lentivirus, human immunodeficiency virus (HIV), varicella-zoster virus, herpes simplex virus, cytomegalovirus, Japanese encephalitis virus, West Nile virus, Zika virus, Haemophilus influenzae type b, measles virus, mumps virus, rubella virus, respiratory syncytial virus, influenza virus, yellow fever virus, rabies virus, smallpox virus, parvovirus, chikungunya virus, other flaviviruses, other alphaviruses, Corona viruses (e.g., causes COVID-19, or derivative thereof). In some embodiments, one or more polypeptide, protein or fragment thereof, antibody or fragment thereof, small molecule or polynucleotide construct or complex thereof or modified polynucleotide construct or complex encoding at least one therapeutic agent thereof is or encodes at least one antigen specific to one or more bacterial pathogen. In certain embodiments, a bacterial pathogen can include, but is not limited to, Corynebacterium diptheriae, Clostridium tetani, Clostridium botulinum, Bordetella pertussis, Streptococcus pneumoniae, Neisseria meningitides, Salmonella spp., Bacillus anthracis, Yersinia spp., B. pseudomallei (Bp), other bacterial pathogen or the like, or a combination thereof.

In some embodiments and further to paragraphs [0029]-[0050] above, one or more polypeptide, protein or fragment thereof, or polynucleotide construct or complex thereof or modified polynucleotide construct or complex is or encodes at least one therapeutic agent thereof is or encodes at least one antigen specific to one or more of a canine-related microbial pathogen. In accordance with these embodiments, one or more canine-related pathogen can include, but is not limited to, canine parvovirus, canine distemper virus, canine adenovirus, rabies virus, canine parainfluenza virus, canine influenza virus, canine corona virus, measles virus, Bordetella bronchiseptica, Leptospira spp., Borrelia burgdorferi, or a combination thereof.

In some embodiments and further to paragraphs [0029]-[0050] above, one or more polypeptide, protein or fragment thereof, or polynucleotide construct or complex thereof or modified polynucleotide construct or complex is or encodes at least one therapeutic agent thereof encodes at least one antigen specific to one or more of a feline-related microbial pathogen. In accordance with these embodiments, one or more feline-related pathogen can include, but is not limited to, feline herpesvirus 1, feline calicivirus, feline panleukopenia virus, rabies virus, feline leukemia virus, feline immunodeficiency virus, virulent systemic feline calicivirus, Chlamydophila felis, Pasteurella haemolytica, Bordetella bronchiseptica, or a combination thereof.

In some embodiments and further to paragraphs [0029]-[0050] above, one or more polypeptide, protein or fragment thereof, or polynucleotide construct or complex thereof or modified polynucleotide construct or complex is or encodes at least one therapeutic agent thereof encodes at least one antigen specific to one or more of a equine-related microbial pathogen. In accordance with these embodiments, one or more equine-related pathogen can include, but is not limited to, Eastern equine encephalomyelitis virus (EEEV), Western equine encephalomyelitis virus (WEEV), Venezuelan equine encephalomyelitis virus (VEEV), rabies virus, Chikungunya virus, West Nile virus, equine influenza virus, equine herpesvirus, Streptococcus equi equi, Clostridium tetani, Neorickettsia risticii, Clostridium tetani, or a combination thereof.

In some embodiments and further to paragraphs [0029]-[0050] above, one or more polypeptide, protein or fragment thereof, polynucleotide construct or complex thereof or modified polynucleotide construct or complex encoding at least one therapeutic agent thereof is or encodes at least one antigen specific to one or more of a bovine-related microbial pathogen. In accordance with these embodiments, one or more bovine-related pathogen can include, but is not limited to, bovine herpesvirus, parainfluenza type 3 virus, bovine viral diarrhea virus, bovine respiratory syncytial virus, Clostridium chauvoei, Clostridium septicum, Clostridium novyi, Clostridium perfringens type C, Clostridium perfringens type D, Pasteurella haemolytica, Clostridium haemolyticum, Chikungunya virus, or a combination thereof.

In some embodiments and further to paragraphs [0029]-[0050] above, one or more polypeptide, protein or fragment thereof, polynucleotide construct or complex thereof or modified polynucleotide construct or complex encoding at least one therapeutic agent thereof is or encodes at least one antigen specific to one or more of an avian-related microbial pathogen. In accordance with these embodiments, one or more avian-related pathogen can include, but is not limited to, avian herpesvirus, Marek's disease virus, reovirus, avian encephalomyelitis virus, avian influenza virus, avipoxviruses, chicken anemia virus, Pasteurella multocida, Newcastle disease virus, Riemerella anatipestifer, duck herpesvirus 1, duck hepatitis virus, or a combination thereof.

In some embodiments and further to paragraphs [0029]-[0050] above, one or more polypeptide, protein or fragment thereof, polynucleotide construct or complex thereof or modified polynucleotide construct or complex encoding at least one therapeutic agent thereof is or encodes at least one antigen specific to one or more of microbial pathogens. In accordance with these embodiments, one or more microbial pathogens can include, but is not limited to, one or more of Cryptococcus spp., Aspergillus spp., Blastomyces spp., Candida albicans, Paracoccidioides spp., Sporothrix spp., Histoplasma capsulatum, Pneumocystis jirovecii, Coccidioides immitis, or a combination thereof.

In yet other embodiments and further to paragraphs [0029]-[0050] above, the pathogenic microbial agent can be a toxin, such as ricin toxin or botulinum toxin.

In yet other embodiments and further to paragraphs [0029]-[0050] above, inactivated or attenuated pathogens (e.g. live, attenuated viruses) can be spray-dried into thermostable glassy microparticles for ALD coating and dissolution assay testing disclosed herein. In accordance with these embodiments, inactivated (or killed) viruses or virus particles, bacteria, or other pathogens can be inactivated by any means known in the art, for example, chemically or by heat and incorporated into thermostable glassy microparticles. Non-limiting examples of inactivated pathogens can include, but are not limited to, inactivated whole-cell pertussis (inactivated Bordetella pertussis), Salmonella typhi, and inactivated polio virus. Live, attenuated viruses or bacteria can similarly be incorporated into thermostable glassy microparticles. Non-limiting examples of attenuated viruses and bacteria that can be incorporated into thermostable glassy microparticles can include measles virus, mumps virus, rubella virus, influenza virus, chicken pox virus, smallpox virus, polio virus, rotavirus, flaviviruses (e.g. dengue virus, yellow fever virus), rabies virus, typhoid virus, Mycobacterium bovis, Salmonella typhi, and Rickettsia spp, or other pathogenic bacteria.

In some embodiments and further to paragraphs [0029]-[0050] above, ALD coated therapeutic agent-containing particles described herein can be include particles of use as vaccines for animals such as household pets, horses, livestock or other mammals, birds, reptiles and amphibians for example. In accordance with these embodiments, ALD coated therapeutic agent-containing particles for use in dissolution assays disclosed herein can be one for application, for example, to a dog (canine), a cat (feline), a horse (equine), cattle (bovine), a goat (hircine), a sheep (caprine), or poultry (e.g., chicken, turkey, duck, goose).

In other embodiments and further to paragraphs [0029]-[0060] above, a liquid formulation containing one or more polypeptide, protein or fragment thereof, one or more polynucleotide construct or complex thereof or modified polynucleotide construct or complex encoding at least one therapeutic agent can be exposed to a spray-drying process disclosed herein and is essentially dried when spray drying is completed, creating a stabilized agent (e.g. glassy particle for example, when in the presence of one or more glassy agents). In accordance with these embodiments, the essentially dry composition can then be coated or encapsulated (e.g. completely encapsulated with minimum to no pinholes or cracks) with at least one layer of coating material (e.g., metal oxide or metal alkoxide) or encapsulation. In certain embodiments, the essentially dry composition can be coated by an atomic layer deposition (ALD) coating process known in the art (e.g., PCT/US2017/019163 incorporated herein in its entirety for all purposes). In some embodiments, each layer of the one or more outer coating layers encasing at least one therapeutic agent-containing essentially dry particle can include aluminum oxide, an aluminum alkoxide (e.g., alucone), silicon dioxide (SiO₂), titanium dioxide (TiO₂), or silicon nitride (Si₃N₄), zinc conjugate (Zn), alone or in a suitable combination composition. In accordance with these embodiments, the outer coating layer(s) can be about 0.1 nm to about 20 nm in thickness. In certain embodiments, the essentially dry microparticles can include a number of outer coating layers sufficient to delay release or provide a timed-release of the at least one agent from the central or innermost agent-containing microparticle and if desired, additional layers added for time-release or introducing at least complex encoding a second therapeutic agent. In accordance with these embodiments, dissolution assays disclosed herein can be used to assess target coating layer numbers for a selected therapeutic agent's timing for release in vivo for improved accuracy and efficiency of delivery.

In other embodiments and further to paragraphs [0029]-[0061] above, the essentially dry composition or partially or completely coated or encapsulated essentially dry composition can be reconstituted in any dissolution buffer contemplated herein for further analysis. In accordance with these embodiments, a dissolution buffer can include a sterile or microbial free buffer (e.g. phosphate, tris or other similar buffer) for analysis and assessment of optimal coating layers and coating compositions for a target therapeutic agent, immunogen, or therapeutic contemplated herein.

In other embodiments and further to paragraphs [0029]-[0062] above, essentially dry (e.g., post spray-dried) formulations, compositions, or particles containing at least one therapeutic agent including, but not limited to, a polynucleotide, polynucleotide construct, polypeptide, protein, chimera, chemical therapeutic, small molecule, other therapeutic or immunogenic agent, or complex thereof, or mixture thereof, or modified version thereof can be encapsulated or coated with one or more ALD applied coating layers to produce a timed-release formulation. In certain embodiments, the coated or encapsulated therapeutic agent-containing particles can be for a timed-release delivery as primary and/or boost doses for a single administration. In accordance with these embodiments, one, two, three, four, five, ten, twenty, thirty, forty, a hundred, two hundred, five hundred, a thousand or any number in between or more than a thousand coating layers can encase the particles where the coating layers dissolve at a predetermined interval or time in a subject once administered and this dissolution is studied and improved using in vitro dissolution compositions and methods disclosed herein.

In other embodiments and further to paragraphs [0029]-[0063] above, coated particles analyzed by dissolution compositions and methods disclosed herein can be used as a vaccine against one or more pathogens, or against cancer, or in delivery of an ALD coated therapeutic agent-containing formulation for gene therapy, for example. In accordance with these embodiments, dissolution compositions and methods disclosed herein can be used to provide optimal conditions for release such as ALD layering composition and number of layers for such a use.

In some embodiments and further to paragraphs [0029]-[0064] above, molecular deposition techniques can be used to apply nanometer-thick coatings of inorganic, organic, or glass metallo-organic materials on the surface of therapeutic agent-containing microparticles contemplated herein. In certain embodiments, coating or sequestering layer can be an aluminum-based material including, for example, an aluminum oxide or an aluminum alkoxide (e.g., alucone). In accordance with these embodiments, the aluminum-containing material can be deposited on or applied to the surface of the one or more therapeutic agent-containing microparticles to coat or sequester the one or more therapeutic agent-containing microparticles in up to 20, up to 40, up to 100, up to 150, up to 250, up to 300, up to 500 or more layers of the aluminum or other metal oxide or metal alkoxide-containing material to form encased therapeutic agent-containing microparticles.

In some embodiments and further to paragraphs [0029]-[0065] above, metal oxide or metal alkoxide-based coating layers can be used as a coating applied to particles having at least one therapeutic agent and at least one glass-forming agent (therapeutic agent-containing microparticles). In accordance with these embodiments, one or more layers of coating can be formed by coupling trimethyl aluminum to hydroxyl groups of therapeutic agent-containing microparticles, a layer of amine groups can be formed by coupling for example, ethanolamine to the layer of metal oxide- or metal alkoxide-containing material, and a second layer of hydroxyl groups can be formed by coupling for example, maleic anhydride to available amine groups. In this example, it is an ABC-type reaction, for example, this process can be self-limiting and can be used to deposit molecular layers of the material on the therapeutic agent-containing microparticles. For example, hydroxyl groups on the substrate (e.g., therapeutic agent-containing microparticles) can react with trimethyl aluminum, ethanolamine then reacts, leaving terminal amine groups on the surface, and available maleic anhydride reacts with terminal amine groups, regenerating a surface of hydroxyl groups. This ABC-type molecular deposition process can be repeated to provide additional layers as desired and can be used to deliver, for example, 1, 2, 3, 4, 5 or 6 doses of a therapeutic agent to a subject in a single administration depending on the composition or make-up of the ALD coated or sequestered one or more therapeutic agent-containing microparticles. In other embodiments, various chemical substitutes can be used in these coating or sequestering processes (e.g., alternative sources for the aluminum, amine, and/or hydroxyl groups), as would be recognized by one of ordinary skill in the art and based on the present disclosure.

In certain embodiments and further to paragraphs [0029]-[0066] above, ALD coated therapeutic agent-containing particles can analyzed by dissolution compositions and methods for prolonged stability and integrity of at least a few hours, about a week, several weeks, at least one month, or more at elevated temperatures and adapted conditions of dissolution buffers disclosed; optionally, where elevated temperatures are at least above room temperature.

In some embodiments and further to paragraphs [0029]-[0067] above, stabilized therapeutic agent-containing microparticles (e.g., essentially dry) and/or coated or encapsulated particles disclosed herein can be a single administration composition capable of eliciting an immune response to two or more different encoded therapeutic agents. In accordance with these embodiments, the two or more different encoded therapeutic agents can be included in the same coated particles, or in separate coated particles. In other embodiments, when the two or more different therapeutic agents or encoded therapeutic agents are contained in separate particles, each of the same or different encoded therapeutic agent-containing particle can include the same or different therapeutic agent as desired and optimum coating for the one or more same or different coated therapeutic agent-containing particles can be determined by compositions and methods disclosed herein.

In some embodiments and further to paragraphs [0029]-[0068] above, therapeutic agent-containing particles disclosed herein can include at least a one encoded therapeutic agent and/or immunogenic agent in one layer and at least a second of the same or different therapeutic agent and/or immunogenic agent or antigen in at least a second layer of a single ALD coated particle. In accordance with these embodiments, dissolution compositions and methods disclosed herein can be used to assess an outer most ALD coated therapeutic agent-containing layer of a particle and subsequently at least a second inner layered ALD coated therapeutic agent-containing layer of a particle; optionally wherein the second therapeutic agent containing layer is the core of the particle. In accordance with these methods, optimal coating layer consistency and number can be assessed for ALD coated microparticles with more than one layer of therapeutic agent in a single assay at reduced time for assessing in vivo delivery of each of the more than one layer of therapeutic agent. In accordance with these compositions and methods, single particles can include ALD coated two, three, four or more therapeutic agents for assessing dissolution rates. In certain embodiments, two or more therapeutic agents can be positioned in the same layer of an ALD coated particle and in other embodiments, the two or more therapeutic agents can be positioned in different layers of an ALD coated particle.

In some embodiments and further to paragraphs [0029]-[0069] above, an ALD coated therapeutic agent or encoded therapeutic agent can be directed to a toxin, such as ricin toxin or botulinum toxin and dissolution rates for these coated agents can be assessed herein. In accordance with these embodiments, the immunogenic composition can be of use, for example, as a treatment for humans or for a pet or other animal such as a dog (canine), a cat (feline), a horse (equine), cattle (bovine), pig (porcine), a goat (hircine), a sheep (caprine), or poultry (e.g., chicken, turkey, duck, goose).

In some embodiments and further to paragraphs [0059]-[0070] above, ALD coated therapeutic agent containing particles or encoded therapeutic agent-containing particles described herein can be of use to treat, prevent or ameliorate a health condition in a human and dissolution rates for these coated agents can be assessed herein to improve delivery and outcome of treatment or amelioration of a condition. In certain embodiments, a therapeutic agent disclosed herein can be used to deliver one or more therapeutic agent containing compositions to a human such as an infant or child or adolescent or adult, including but not limited to vaccines for varicella-zoster (chicken pox), diphtheria, Haemophilus influenzae type b (Hib), HPV (all pathogenic types), hepatitis A, hepatitis B, influenza, measles, mumps, pertussis, polio, pneumococcal disease, rotavirus, rubella, and tetanus. In other embodiments, immunogenic agent-containing particles described herein may be used to deliver one or more immunogenic compositions to a human pre-teen or teen, including but not limited to vaccines for influenza, tetanus, diphtheria, pertussis, human papillomavirus, meningococcal disease, hepatitis B, hepatitis A, polio, measles, mumps, rubella, and varicella-zoster. In yet other embodiments, ALD-coated therapeutic agent containing particles or encoded therapeutic agent-containing particles can include those against travel-related diseases or any pathogenic organism thereof in humans, including but not limited to, hepatitis A, hepatitis B, paratyphoid fever, meningococcal disease, yellow fever, Zika infection, rabies, dengue fever, rabies, Chikungunya disease, typhoid fever, malaria, covid or covid variant, and Japanese encephalitis or other flavivirus or alphavirus-related condition and dissolution rates for these coated agents can be assessed herein.

In some embodiments and further to paragraphs [0029]-[0071] above, coating layers other than aluminum-based coating layers can be used in order to coat or sequester the therapeutic agent-containing particles. In accordance with these embodiments, non-aluminum coating layers including, but not limited to, silicon dioxide (SiO₂), titanium dioxide (TiO₂), or silicon nitride (Si₃N₄) or zinc can be used either in combination with aluminum-based coating layers, or alone to the exclusion of aluminum-based coating layers. With each type of material having different characteristic dissolution times, layers of different materials can be deposited on the particles to vary the temporal release of the at least one therapeutic agent from a coated particle layer and/or a coated particle's core. In some embodiments, one or more aluminum-based layers can be introduced, followed by one or more layers of a different material. In accordance with these embodiments, different materials may dissolve more slowly than the aluminum-based coating layer. Using other materials for coating the particles, can reduce the number of aluminum-based layers necessary to provide for a given release time, minimizing the amount of aluminum per dose for reduced adverse effects of alumina as appreciated by one of skill in the art.

In certain embodiments and further to paragraphs [0029]-[0072] above, based on observations using dissolution compositions and methods disclosed herein, ALD methods can be optimized for a particular situation or condition. For example, antigens against a pathogenic organism incorporated into particles can have variable thermostability, and therefore might not be amenable to conditions of higher ALD temperatures due to this vulnerability. In some embodiments, molecular deposition can occur under vacuum conditions. By performing atomic layer deposition under vacuum conditions, coating layers can be applied at reduced temperatures thereby reducing adverse effects of higher temperatures on target agents. In certain embodiments, vacuum conditions required for deposition can be minimal. In some embodiments, an ALD coating process can occur under a mild vacuum of about 0.1 atmospheres. In other embodiments, ALD coating processes can also include incorporation of mechanical agitation devices that can lead to shorter cycle times for deposition of the material by providing uniform distribution of powders and reactants within an ALD reactor. It is contemplated herein that any ALD coated therapeutic agent containing particle can be analyzed using dissolution compositions and methods disclosed herein.

### Kits

Other embodiments and further to paragraphs [0029]-[0073] above, provide kits of use with compositions and methods disclosed herein. In certain embodiments, a kit can contain one more dissolution buffers for assessing optimal coating layers in an in vitro setting. Kits can further include one or more of an aqueous composition or essentially dry and/or coated particles and at least one container.

In other embodiments and further to paragraphs [0029]-[0074] above, kits are contemplated of use for compositions, and methods described herein. Kits can be portable. In certain embodiments, kits can be used to transport to and be used in remote areas. In some embodiments, kits can include a dissolution composition disclosed herein, at least one container and optionally, directions for use of the kit, of use to analyze any ALD-coated therapeutic agent containing particle.

In some embodiments and further to the preceding paragraphs, kits can include one or more suitable containers, for example, vials, tubes, mini- or microfuge tubes, test tube, flask, bottle, syringe, or other container. Where an additional component or agent is provided, the kit can contain one or more additional containers into which this agent or component may be placed. Kits herein will also typically include a vessel for containing the dissolution buffers and or coated particles, and any other reagent container. In some embodiments, dissolution assays disclosed herein can be performed in microtiter wells or microtiter plates, for example, to obtain rapid ALD quality analysis using reduced concentrations of ALD-coated particles disclosed herein.

It will be recognized that the embodiments described herein can be applied to all ALD coated therapeutic-agent containing particles. For example, small molecule drugs (e.g., anti-cancer agents) and biologics can be similarly coated, and dissolution rates determined by compositions and methods disclosed herein. Coating layers provide for a level of temporally controlled release desirable with agents. Coating layers can serve to reduce exposure to moisture, reducing degradation. Coating layers can function to protect water-soluble drug formulations or other moisture sensitive agents from degradation or dissolution until desired exposure to a subject after administration. Further, the embodiments can be used in applications outside of therapeutics for assessing dissolution of a targeted agent. For example, coating layers can be applied to diagnostic markers or agents and dissolution analysis can be performed to assess inflection or optimum layering of the diagnostic marker or diagnostic agent. The coatings can allow delayed release of the marker, allowing sufficient trafficking/uptake time. This can be beneficial where the marker has a limited half-life. Understanding optimum release time of these markers can be critical in timing for increased accuracy of diagnosis based on timing of release of the marker(s). These coated particles can be optimized for targeted delivery using dissolution studies disclosed herein.

### EXAMPLES

The materials, methods, and embodiments described herein are further defined in the following Examples. Certain embodiments are defined in the Examples herein. It is understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1

In one exemplary method, an accelerated in vitro method to accurately assess quality of ALD coatings and discriminate dissolution behavior based on the type of ALD coating was developed. Specifically, in certain exemplary methods, 10 mg/ml ALD coated therapeutic agent-containing particles were suspended in a for example, 100 mM potassium phosphate, pH 6.5 dissolution buffer to form a mixture. The mixture was heated, for example, to 50 °C, to accelerate release and reduce experimental duration, optionally while mixing the sample continuously to promote homogeneity. Then, the sample was incubated, and particles separated from the dissolution at desired time points to draw therapeutic agent-containing samples from the supernatant. Dissolution was allowed to continue by replacing sample volume, resuspending particles, and continuing incubation under dissolution conditions.

### Example 2

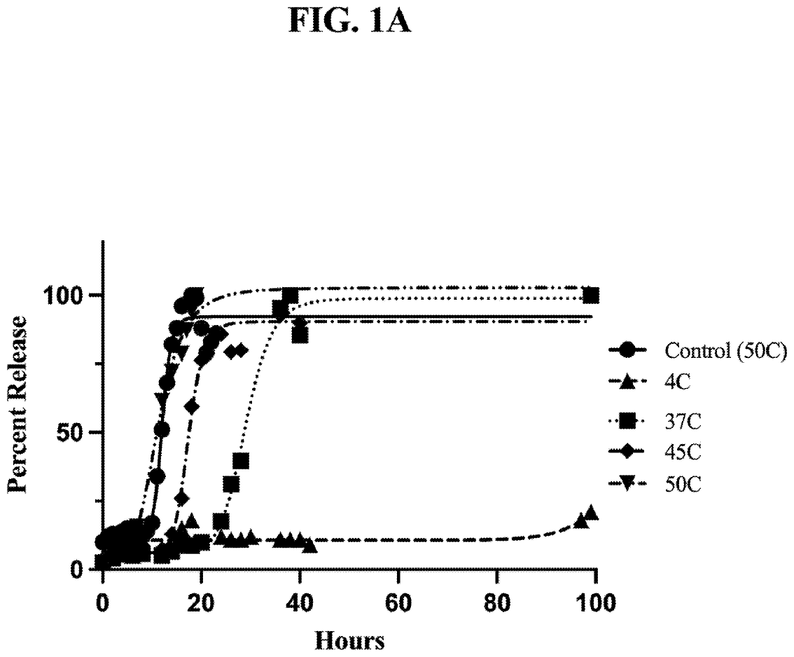

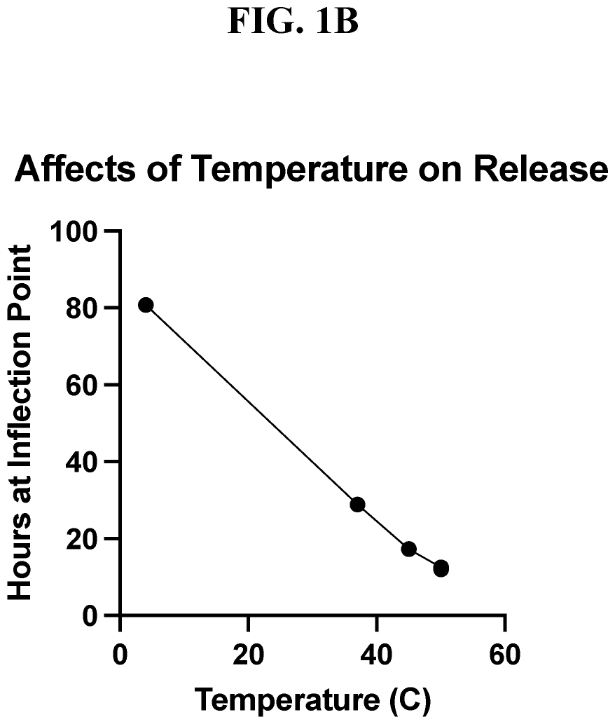

In another exemplary method, the dissolution protocol described in Example 1 was repeated on 100 coat ALD-coated material (ALTA™ material, representative protein spray dried into glassy particles) in 0.1 M Potassium Phosphate buffer (pH 6.5) at different temperatures (4 °C, 37 °C, 45 °C, and 50 °C). In this example, amount of release of the sequestered test material was measured and was plotted (see for example, FIG. 1A). It was discovered that the ALD-coated material (ALTA™ material, a representative coated therapeutic agent, ovalbumin) dissolved faster and released the sequestered test material faster as the experimental temperature increased (See for example, FIG. 1B). This suggests that temperature of these assays is a significant influence on dissolution rate of the coated particles. While these dissolution assays can be performed at a lower temperature, this can be done at the expense of the experimental duration where the experiment at a lower temperature has extended analysis time and increased time to essentially total release. A correlation exists where a reduced temperature dramatically increased the duration of release of the sequestered test material. It was observed that agitation could be useful in some dissolution assays to ensure homogeneity of release but was not found to be a requirement when using a low-viscosity dissolution composition. As disclosed herein, inflection point will still exist and the relationship will be predictable, the time to reach the inflection increases with decreasing temperature. It is observed that these in vitro assays provide conditions which generate predictable and differentiating/discriminating release between types of ALD coatings and happens to also have a relationship with in vivo behavior.

### Example 3

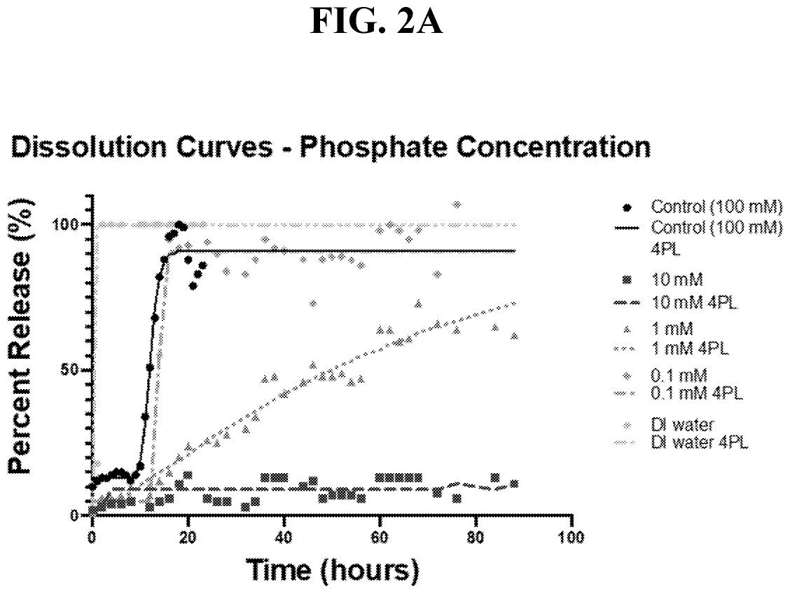

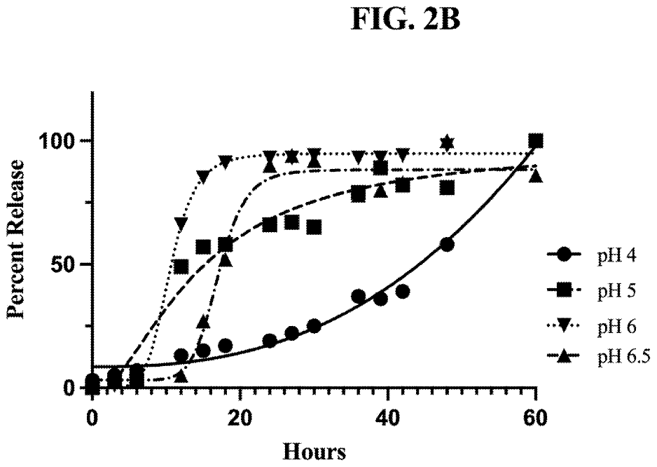

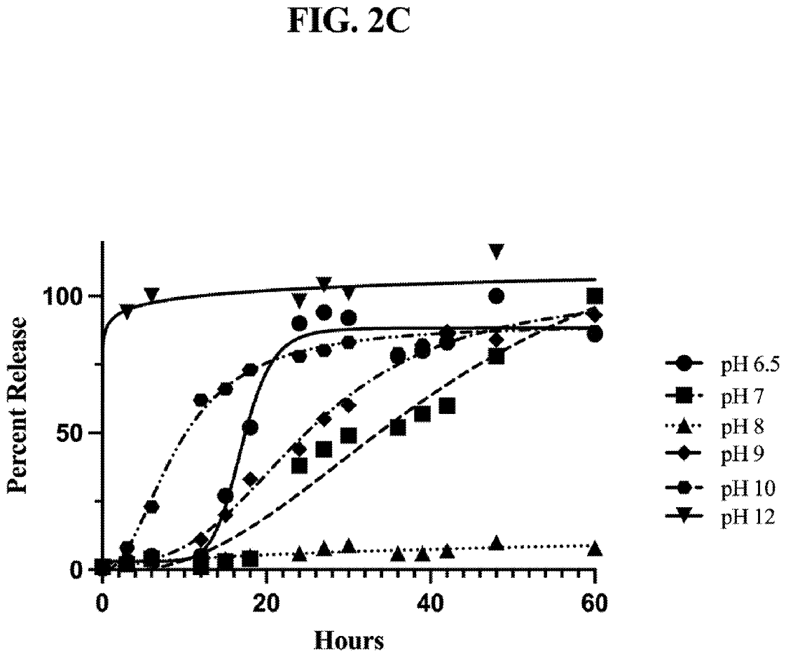

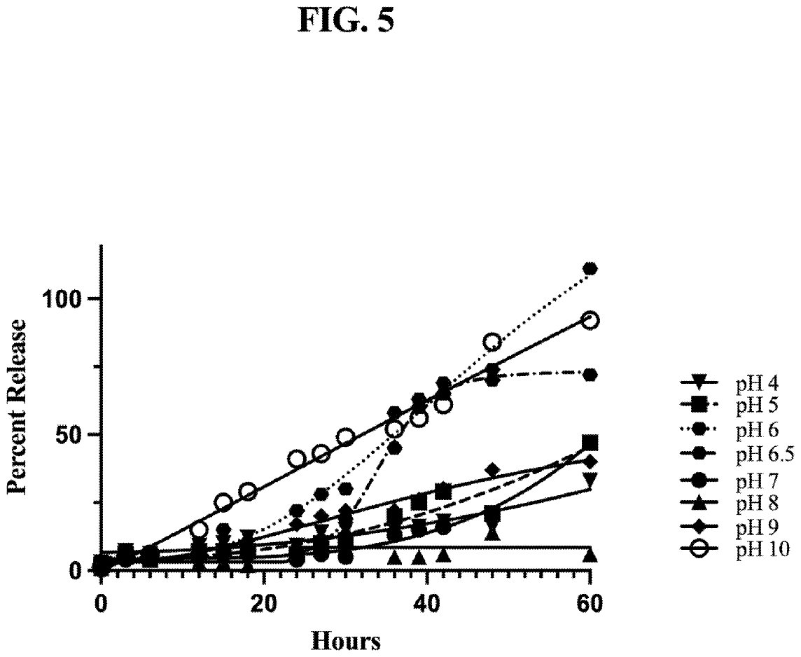

In another exemplary method, the dissolution protocol described in Example 1 was repeated using phosphate buffers with varying phosphate concentrations (e.g., 100 mM, 10 mM, 1 mM, 0.1 mM, or 0 mM potassium phosphate), and/or varying pH (e.g., pH 4, pH 5, pH 6, or pH 6.5, pH 7, pH 8, pH 9, pH 10 or pH 12). In one experiment, effects of increasing phosphate concentration on dissolution is represented in FIG. 2A. It was observed that dissolution is significantly reduced in potassium phosphate buffers from 1.0-10.0 mM but can be significantly accelerated at both higher concentrations of about 10.0 mM to about 1.0 M and lower concentrations less than 1.0 mM. These experiments surprisingly identified a concentration of about 0.1 M as one preferred concentration under conditions disclosed herein. The concentration of phosphate buffer was held constant at 0.1 M and the effects of increasing pH on dissolution is also illustrated, for example, in FIG. 2B (pH 4-pH about 6.6) and FIG. 2C (about pH 6.5-12). It was observed that pH of the dissolution compositions can play a significant role in metal oxide solubility/dissolution, phosphate complexation efficiency, and charge and, also, dissolution rate of ALTA™ coated agent dissolution. This suggests that, in some exemplary methods, phosphate-induced surface rearrangement could play an important role in the mechanism and/or tuning of ALTA™ dissolution (See for example FIGS. 2B and 2C and FIG. 5). In 2B and 2C, the same lot of 100-coat powder that was ran in 100 mM potassium phosphate at different pH at 50 °C. The only aspect that was changed was the pH of the potassium phosphate. As represented in FIG. 5, the same 100-coat lot of material was used in 100 mM sodium phosphate at different pH at 50 °C. The difference here from FIGS. 2B and 2C is that the runs were done in sodium phosphate instead of potassium phosphate. While use of sodium phosphate works in these assays, it was observed that inclusion of potassium phosphate demonstrated superior results compared to sodium phosphate, allowing for specificity between different numbers of coating layers and different types of coatings. Therefore, it was demonstrated that phosphate/pH driven dissolution differed for sodium vs. potassium phosphate buffers, this could be due to these ions have different affinity for phosphate.

### Example 4

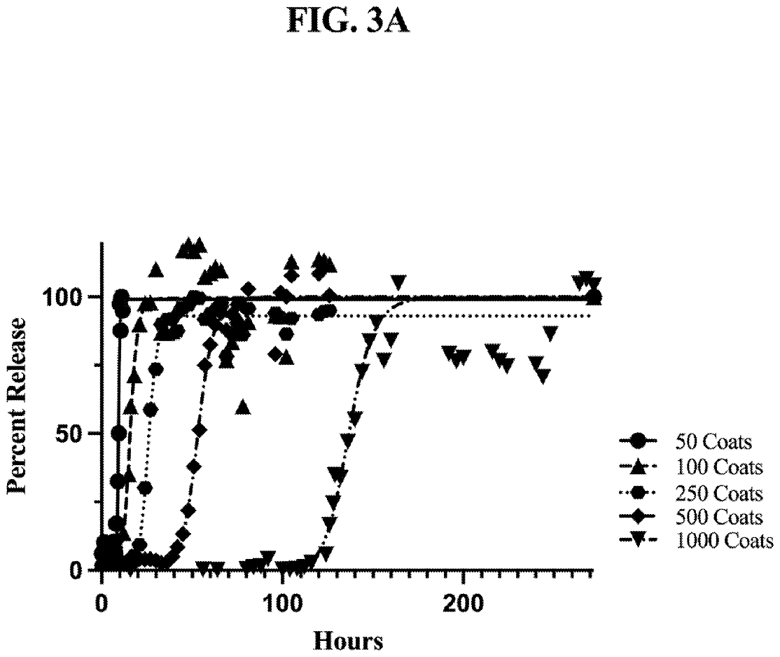

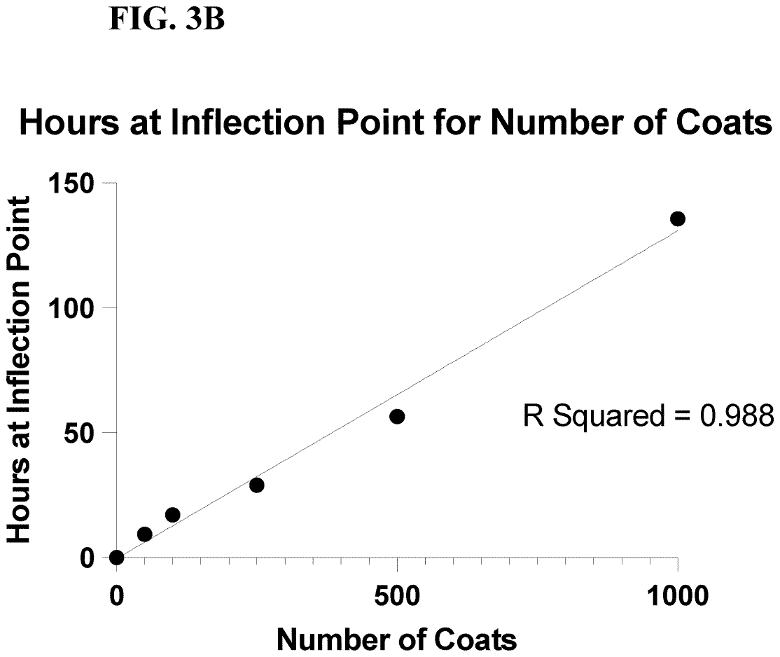

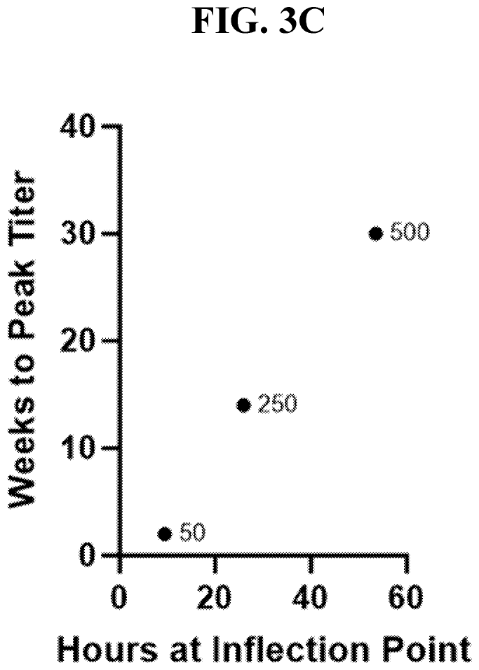

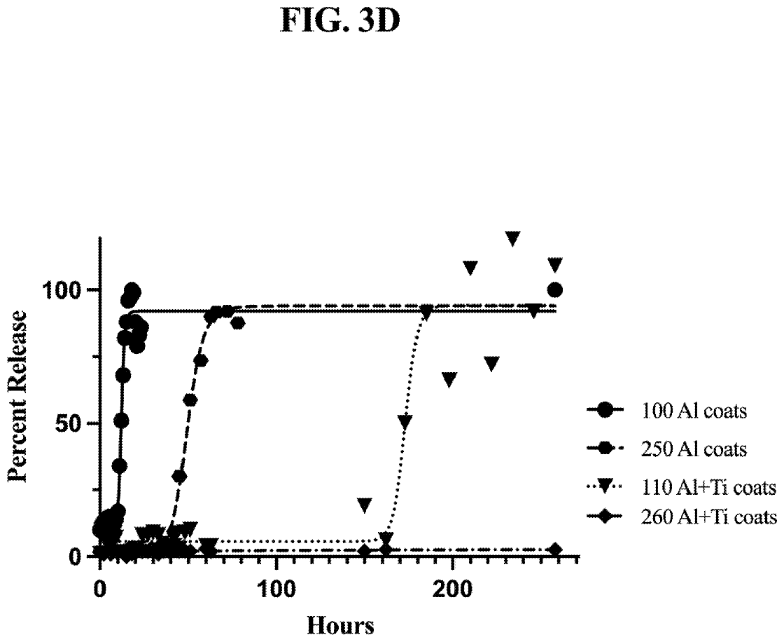

In another exemplary method, the dissolution protocol described in Example 1 was repeated on ALD-coated material (ALTA™ material, exemplary material ovalbumin representative of any therapeutic agent contemplated herein) with varying coat numbers (50-1000 coats). A 0.1 M potassium phosphate buffer at pH 6.5 was used under standard accelerated dissolution conditions. FIGS. 3A-3B demonstrates effects of increasing coat numbers on the percent released at a pre-determined times (See FIG. 3A) and time to release as it correlates with the number of coatings on particles (See FIG. 3B). For example, it is demonstrated that inflection point which is the point of majority of release correlates linearly with the applied number of coating layers to the targeted therapeutic agent(s). In another exemplary method, experiments were performed to assess how release profiles relate to antibody production against the encased test agent in vivo. Animals were inoculated with materials having 50, 250 or 500 coats of the ALD-coated material (ALTA™ material, exemplary material ovalbumin) and measured for antibody titer. The number of weeks to peak titer in vivo was plotted relative to the hours at inflection point in vitro for each coat number and is illustrated for example, in FIG. 3C which demonstrates that the time to generate antibodies in vivo correlated with the duration of dissolution in vitro. Therefore, dissolution assays disclosed herein can be used to further predict or project antibody production to a particular immunogenic agent contained in ALD coated particles and/or further understand active or real-time release in vivo. In addition, use of dissolution assays disclosed herein provided significant improvements in predicting optimal release of a therapeutic agent relative to number of coating layers, etc.

In another exemplary method, dissolution profiles of ALD-coated material (ALTA™ material, exemplary material ovalbumin) having Aluminum-only and Aluminum+Titania coats were examined. As illustrated in FIG. 3D, presence of titania with aluminum in coating compositions for test particles resulted in significantly slower dissolution rates of the tested materials compared to alumina only ALD coatings. The lines represent four parameter logistic regression (4PL regression) fits generated by the experimental data. In these exemplary experiments, reduced number of coating layers were identified as equivalent while achieving the same or similar release time using these combination titania and alumina coatings compared to using a composition containing alumina only coated materials. These observations provide support for using this combination as an alternative to reduce alumina exposure and conserve costs and time for coating while getting the same timed-release benefit of the coated therapeutic agent(s).

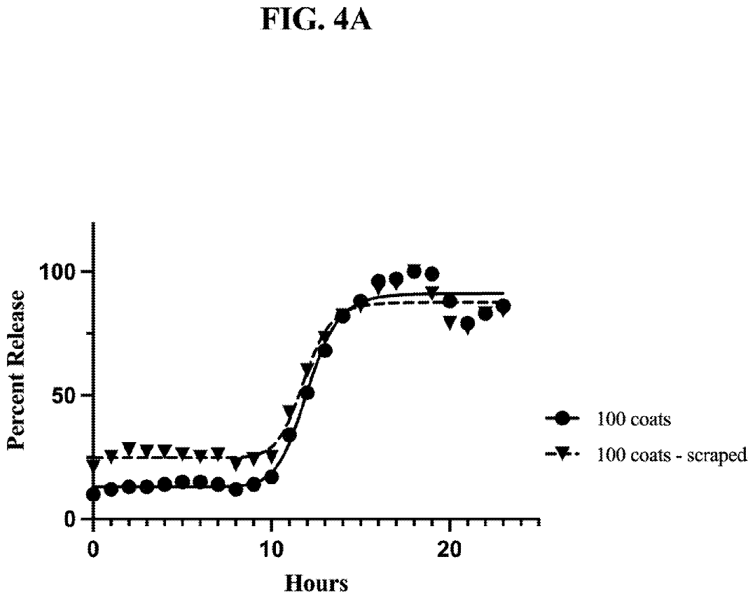

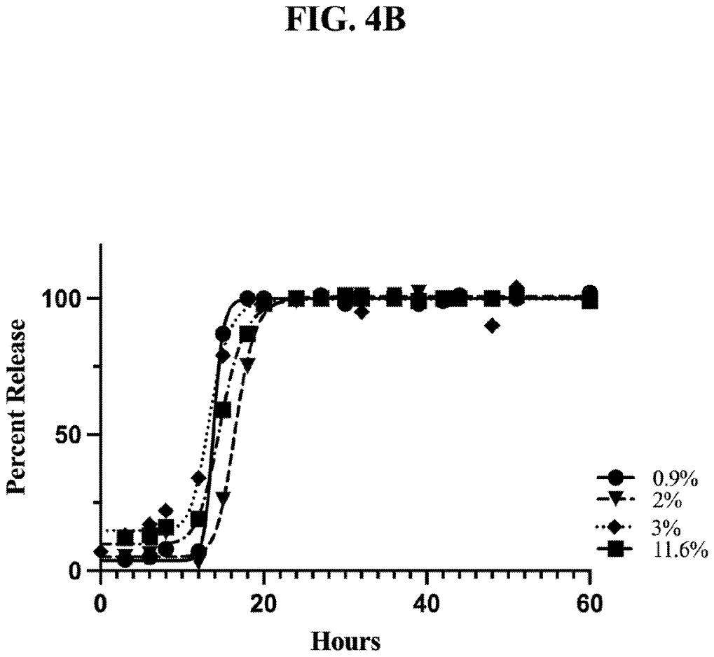

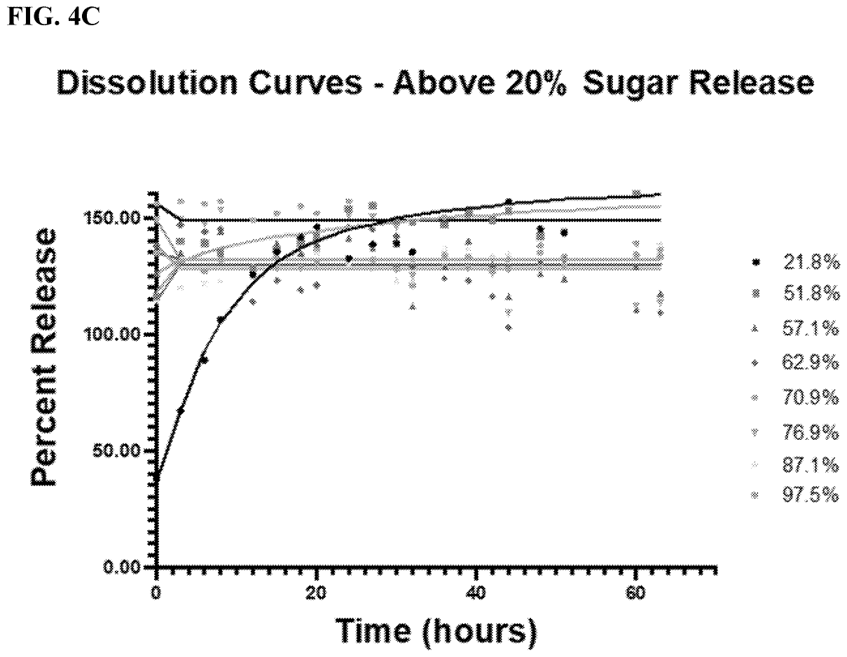

In another exemplary method, experiments were performed to determine coat quality using a basic sugar-release assay. In this assay, ALD-coated material (ALTA™ material) are placed in distilled water at room temperature and incubated rocking for 10 minutes, after which the sample is centrifuged, and the supernatant is collected and evaporated to determine the quantity of released sugar. Sugar release varies for different coat numbers, but powders which release less than 15% of the total sugar content by this assay appear to have acceptable encapsulation protection and coat quality. For this example, the dissolution profile of acceptable particles (e.g., having less than 20% sugar release) or unacceptable particles (e.g., having more than 20% sugar release) was plotted (See FIGS. 4B-4C). As illustrated in FIG. 4B, particles with less than 20% sugar release maintain low initial release and dissolution behavior consistent with previous observations, but FIG. 4C illustrates a high initial release if not instantaneous release for particles having equivalent number of coating layers with sugar release values of greater than 20%. In general, these data demonstrate that the disclosed dissolution assay was able to profile and distinguish acceptable from unacceptable coated particles for quality control purposes and more accurate therapeutic agent delivery.

### Example 5

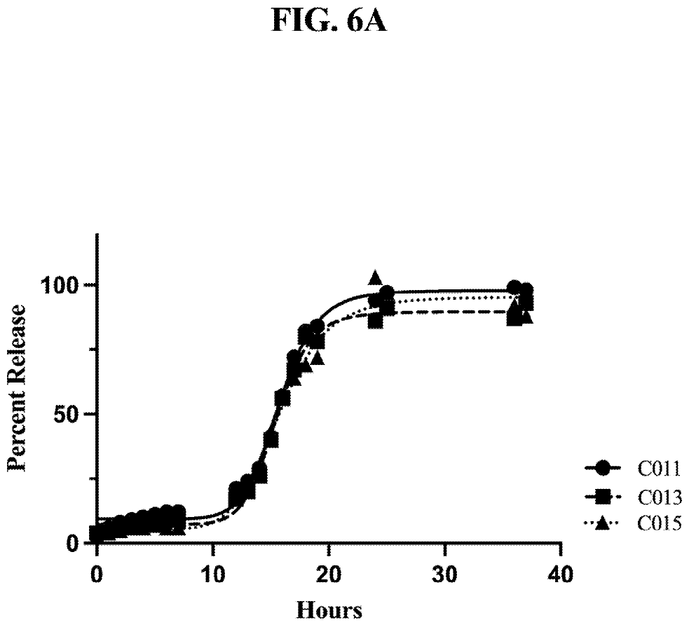

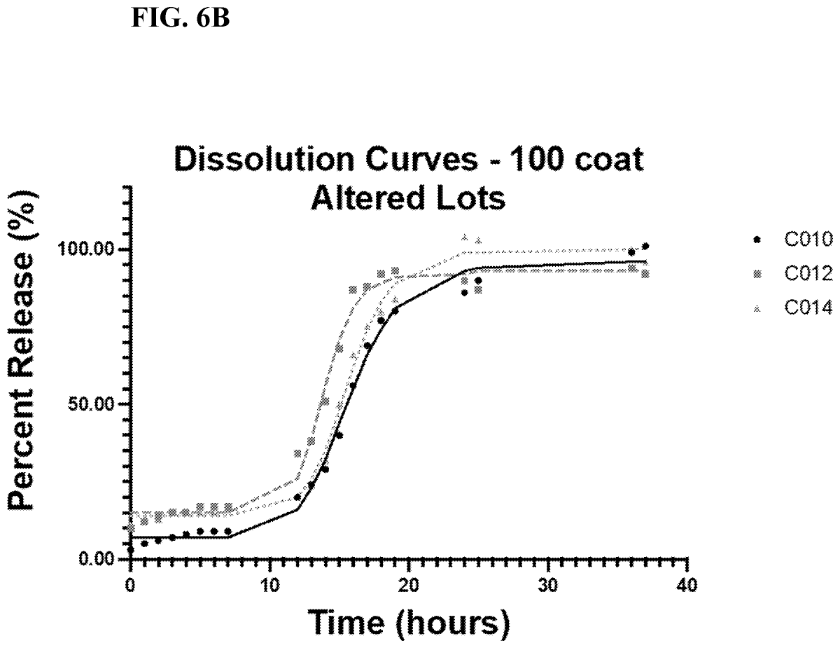

In another exemplary method, dissolution assay variability was investigated using three equivalent lots (FIG. 6A) made using standard ALD-coating methods disclosed herein where sufficient amounts of ALD reactant precursors are used along with sufficient purge time in between the precursors. In another exemplary experiment, three independent lots made with slightly altered deficient ALD-coating methods (FIG. 6B) were used to ensure the dissolution assay could distinguish materials or inferior materials generated outside of these standard ALD coating methods as evaluated in these experiments. Table 1 illustrates inter-lot variability as modeled by a four-parameter logistic regression using these standard coating methods was negligible or minor when using the standard setpoints of the ALD reactor. Furthermore, the disclosed dissolution assays distinguished lots made using these non-ideal coating methods and conditions, whereby precursors were not delivered in equitable quantities and/or not sufficiently purged between exposures, allowing for undesirable chemical vapor deposition reactions. These assays of the non-ideal coated particles reported a higher initial release (A value) with greater variability than compared to the standard method, as expected based on higher sugar release values (e.g., sugar contained within the coating layers and non-ideal generated coating layers), and significantly higher variability in the hours to the inflection point (C value). The representative results illustrated in this Table 1 demonstrates reproducibility of the dissolution assay and that it has the sensitivity to detect small to significant changes in the ALD coating process for improving manufacturing processes and for use in QC materials.

TABLE 1
Reproducibility of four-parameter logistic regression observations
between lots of altered/non-ideal and standard ALD set points

| | | | | Inter-lot | | | | Inter-lot | |
|---|---|---|---|---|---|---|---|---|---|
| | | | | altered | | | | standard | |
| | | | | setpoints | | | | setpoints | Inter-lot |
| | Altered Lots | | | Average | Standard Lots | | | Average | Average |
| Parameter | C010 | C012 | C014 | (±SD) | C011 | C013 | C015 | (±SD) | (±SD) |
| % Release at | 7% | 15% | 14% | 12% | 9% | 7% | 5% | 7% | 9.5% |
| Time 0 (A) | | | | (±4.4) | | | | (±2) | (±4) |
| Slope (B) | 8.2 | 12.2 | 9.9 | 10.1 | 9.5 | 10.5 | 7.6 | 9.2 | 9.7 |
| | | | | (±2) | | | | (±1.5) | (±1.7) |
| Hours at | 15.6 | 13.9 | 15.7 | 15.1 | 15.7 | 15.5 | 15.7 | 15.6 | 15.4 |
| Inflection Point | | | | (±1) | | | | (±0.1) | (±0.7) |
| (C) | | | | | | | | | |
| % Release after | 96% | 93% | 100% | 96% | 98% | 90% | 95% | 95% | 95% |
| Dissolution (D) | | | | (±3.5) | | | | (±3.6) | (±3.6) |

### Example 6

In another exemplary method, the dissolution assay described in the previous examples was used as quality control assay for ALD-coated material (ALTA™ material, exemplary therapeutic agent, ovalbumin).

In one exemplary method, a dissolution curve was generated for standard versus damaged particle (e.g., 100 coats vs 100 coats, scraped particles). There was a significant difference in the dissolution profile for the damaged particles (See FIG. 4A) suggesting that this dissolution assay can also be used to detect damaged particles.

### Example 7

In other exemplary methods, other buffers were tested in dissolution assays disclosed herein. It was found that use of sulfate buffers at pH 6.5 was too rapid for accurate assessment so alternative parameters are needed to slow down the dissolution of the coated particle layers. It was observed that dissolution was too fast for a 0.1 M sodium citrate buffer by itself at pH 6.5 so modifications to this buffer may be needed to achieve a slower more reliable and reproducible dissolution assay using a citrate buffer.

It was also observed that simulated biological fluids corroborated phosphate concentration experiments. Those with little/no phosphate demonstrated a rapid release of ALD-coated material (ALTA™ material, exemplary material ovalbumin), while those with 1.0-10.0 mM phosphate demonstrated minimal release over the experimental period. It was observed that distilled water and saline solutions demonstrated very rapid release of all ALD-coated material (ALTA™ material, exemplary ovalbumin representative of a therapeutic agent contemplated herein) making it difficult to analyze these materials at these tested conditions. For example, for a 1000 coat alumina ALD-coated material (ALTA™ material), release was observed after about 3 hours in distilled water at 50 °C, whereas comparatively, release was observed after ~170 hours in 0.1 M potassium phosphate at 50 °C (see e.g., FIG. 2A).

### Example 8

In another exemplary method, various buffers for injection were contemplated. While saline rapidly releases ALD-coated material (ALTA™ material, exemplary material ovalbumin) at 50 °C, 50 and 100 coat materials demonstrated no release in phosphate buffered saline (PBS) or saline (+/−6% HES) over 24 hours when conditions were at room temperature or under refrigerated conditions (e.g., 4 °C).

### Example 9

It is noted that these results demonstrate that the disclosed compositions and methods allow for assessment of coat quality of ALD coated particles formed for poor solubility and later release of their contents. Further, this method allows for the interrogation of coat quality and discrimination behavior of particles of different coat numbers and coat compositions. As discussed above, temperature, buffer (e.g., phosphate) concentration and pH each can play a significant role in dissolution behavior.

Compositions and methods disclosed herein are advantageous because they require minimal quantities of material, are performed at an accelerated temperature (e.g., 50 °C), using a buffers disclosed herein (e.g., 0.1 M potassium phosphate buffer, pH 6.5) and can be performed with or without mixing. This enables consistent, specific, relatively rapid assessment of coat quality and dissolution behavior of material which takes upwards of months to release in vivo.

### Example 10

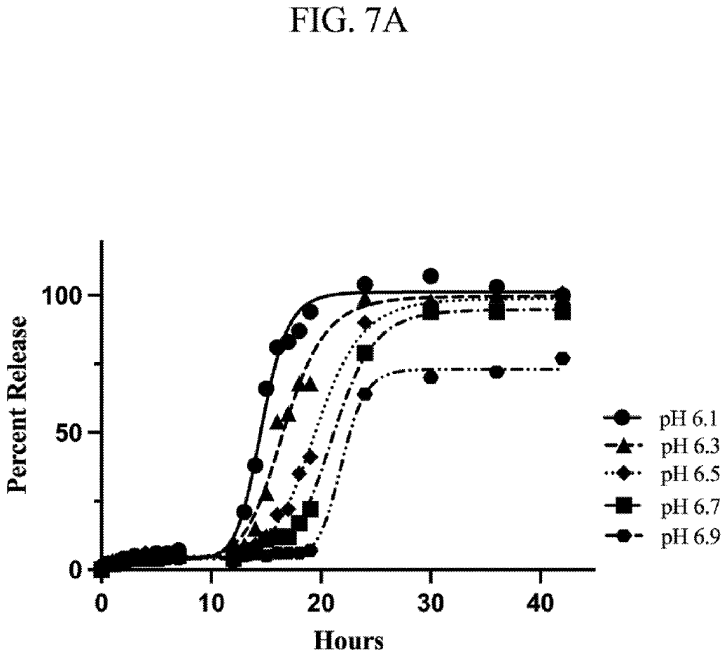

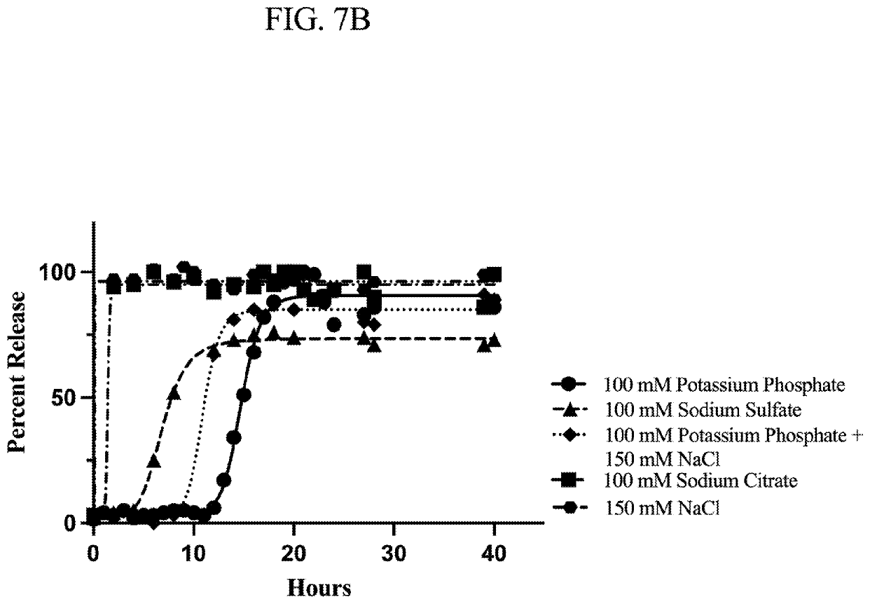

In another exemplary method, plotting curves demonstrated in this figure are representative of the same lot of 100-ALD coated material (e.g., representative polypeptide, ovalbumin). In this example, 0.1 M potassium phosphate dissolution buffer was made at different pHs to examine effect of pH on release kinetics. All samples were heated to 50 °C and in this example, mixed at 950 rpm, continuously until complete. Time points were taken every hour to capture release of the representative agent (e.g. ovalbumin). In these ALD coated agent-containing particles, formulation and protein are being released and measured using an OPA assay. As disclosed herein, the OPA assay is the o-phthalaldehyde assay is a fluorescence-based assay that detects free amines in solution. This applies to the therapeutic agents (APIs) and components in these formulation, when a therapeutic agent is released from dissolution, the OPA assay can be used to measure how much is released at each time point. In accordance with this example and Example 11 below, histidine is detected and measured in these assays. It was demonstrated that pH of a buffer had an effect on agent release whereas demonstrated herein, a more acidic buffer released agent sooner than a more pH neutral buffer. These observations indicate that dissolution kinetics of compositions and methods disclosed herein are dependent on buffer and buffer pH (See for example, FIG. 7A).

### Example 11

In another exemplary method, plotting curves demonstrated in this figure are from the same lot of 100-coat material (e.g., representative polypeptide, ovalbumin). In this example, all samples were heated to 50 °C and agitated at 950 rpm, continuously for the duration of the incubation but can be intermittently. Time points were taken every hour to capture the release of the representative agent (e.g. ovalbumin). In these ALD coated agent-containing particles, formulation and protein are being released and measured using the OPA assay. Different buffers demonstrated varying release kinetics of the representative agent compared to a proven buffer of 100 mM potassium phosphate buffer used as a control in this example Although NaCl and sodium citrate released material the fastest, the release rate was too fast to be able to distinguish small changes in coating and manufacturing for optimization of conditions (See for example, FIG. 7B). Therefore, potassium phosphate at 100 mM demonstrated surprising and superior results to these buffer conditions.

Further, there was a positive correlation between the number of coats and the hours to the inflection point of release (C value produced by four parameter logistic regression) and this positive correlation also existed between the hours to the inflection point of release in vitro and time to maximum antibody titer in vivo for an immunogenic agent or antigen (e.g., ALD coated vaccine). As discussed above, the dissolution assay can further project or predict release of a therapeutic agent in ALD coated therapeutic agent containing particles in vivo.

It is contemplated that the disclosed method can be applied to alumina coatings as well as laminates of different compositions. Further, the detection method is independent of or agnostic to the sequestered or coated therapeutic agent—with experiments successfully applying RP-UPLC, SEC-UPLC, o-phthaladehyde assay and detection of fluorescently-tagged antigen. Further, this method has also been applied to ALD-coated material containing a trimer, a lipid-based adjuvant and placebo for example.

As discussed above, the method can be applied as a quality control assay for ALD coated therapeutic agent containing particles. Further, the dissolution method is amenable to adaptation to a microtiter plate, therefore enabling high throughput screening and/or automation. It is noted that while the data presented above is currently fit to four-parameter-logistic regression, other modeling can be applied (e.g., five, or six-parameter regressions, Avrami, Weibull or other modeling). In addition, materials coated with differing numbers of layers have been tested simultaneously and demonstrated supportive results where the number of layers could be projected for release of their contents based on observations herein.

### Example 12

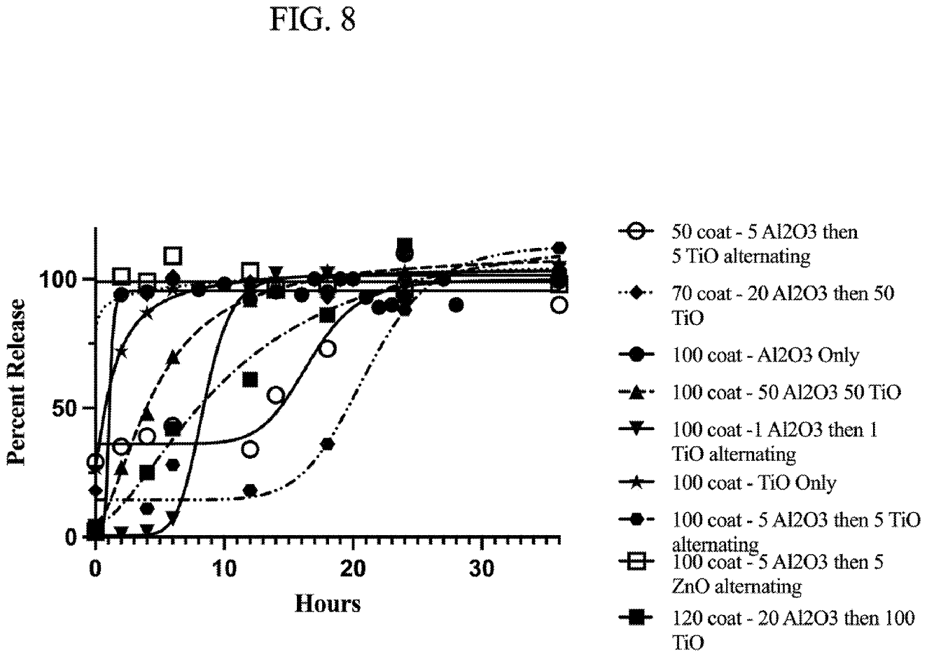

In another exemplary method, plotting curves demonstrated in FIG. 8 are from the same material (e.g., representative polypeptide, ovalbumin) coated with different metals (alumina only, alumina-titania, titania only, and alumina-zinc). In this example, all samples were heated to 50 °C and agitated at 950 rpm continuously. Time points were taken every hour to capture the release of the representative agent (e.g. ovalbumin). In these ALD coated agent-containing particles, formulation and protein are being released and measured using the OPA assay. The buffer used for these powders was 100 mM sodium citrate pH 6.5 to further accelerate the rate of dissolution. The titania laminates took longer to release than the 100-coat alumina-only powder matching to what was seen previously in FIG. 3D but on a time scale that is more manageable for laminate powders. Using this buffer system, laminate powders can be better characterized.

### Example 13

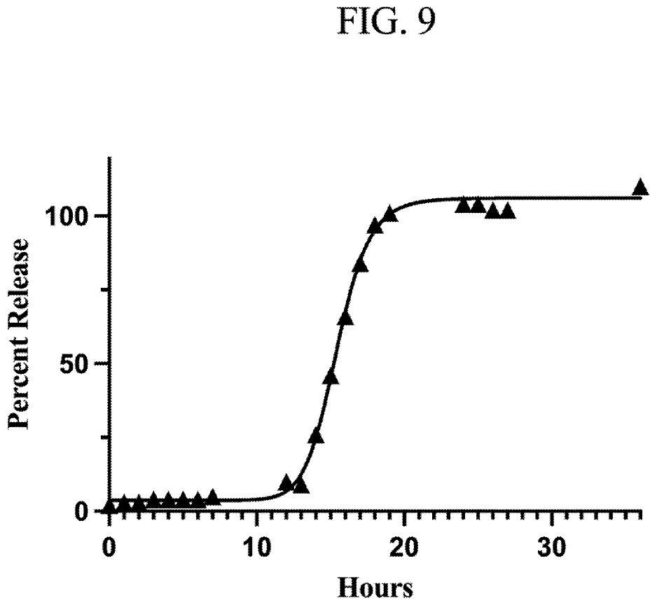

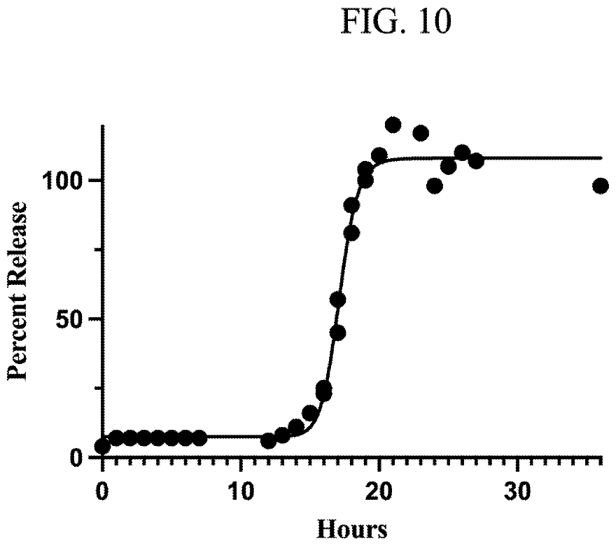

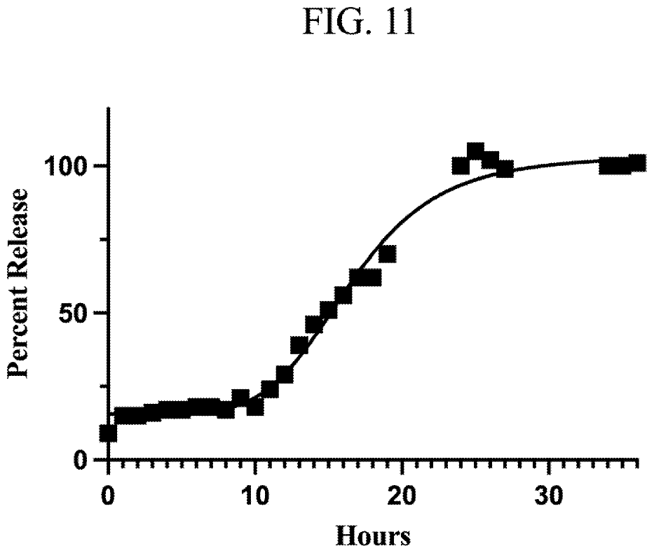

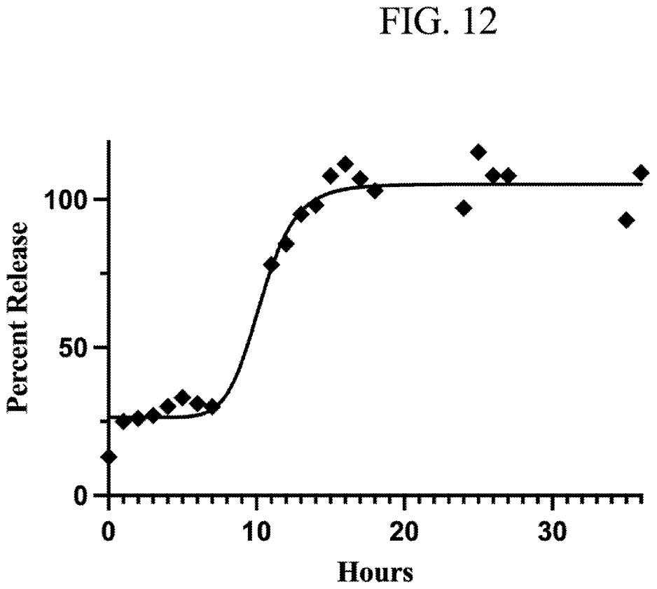

In another exemplary method, plotting curves demonstrated in FIGS. 9-12 are from different materials (e.g. small molecule, glycoconjugate, lipid nanoparticle, and oligonucleotides, as indicated herein) coated with alumina only. In these examples, all samples were heated to 50 °C and agitated at 950 rpm continuously. Time points were taken every hour to capture the release of the representative agent (e.g. small molecule FIG. 9, glycoconjugate FIG. 10, lipid nanoparticle, mRNA-LNP FIG. 11, and oligonucleotide, polypeptide, FIG. 12). In these ALD coated therapeutic agent-containing particles, formulation and active are being released and measured using the OPA assay. The buffer used for these powders was 100 mM potassium phosphate pH 6.5. This data demonstrates that dissolution is agnostic of a sequestered/coated therapeutic agent containing microparticle, and the target agent does not impact the dissolution assay.

As disclosed herein, 4PL regression is one method that can be used to describe dissolution behavior. Either, the 4PL regression can be calculated as y=[D+(A+D)]/[1+(x/C)\hat{ }B] where y is the percent released and x is the number of hours; or regression is one known to those of skill in the art. The four parameters describe the initial percent release (A), the hill slope (B), the time to 50% release (C), and maximum percent release (D). C is the Inflection Point (½ distance between A and D). The A-value can be used to determine the completeness of the coating, the B-value is indicative of particle-to particle variability (more homogeneous particles would be expected to release simultaneously resulting in a sharper slope), the C-value can be used to compare release rates between particles of different cycle numbers or batches, and the D-value captures the extent of release.

As contemplated herein, particles evaluated by these dissolution methods can be described as thermostable, glassy spray dried particle containing antigens/adjuvants or other therapeutic agents, disaccharide, and a high molecular weight outer layer of ALD coating. Ovalbumin as used herein is an exemplary representative or agnostic representative of any other ALD-coated therapeutic agent-containing particles contemplated herein.

### Accelerated Dissolution of ALD Coated Particles In Vitro

It is understood herein that temporal control of immunogenic agent induced immune responses using ALD coated immunogenic agent-containing particles can be challenging. Accelerated in vitro dissolution methods disclosed herein were developed to characterize coatings and can be used to demonstrate that increasing ALD cycle numbers delays immunogenic agent release. For example, time to release in vitro was demonstrated to correlate with the antibody response times in vivo. In certain examples disclosed herein, ALD-coated therapeutic agent containing-particles were suspended at a concentration of 10 mg/mL buffered solution in Lo-bind microcentrifuge tubes and agitated at 50 °C and 950 rpm. In this example, dissolutions were performed at about 50 °C to capture the dissolution profiles of the ALD-coated particles in a reasonable time frame while minimizing the risk of evaporation and microbial contamination. The supernatants were sampled at the indicated times after brief centrifuging to pellet the ALD powder. The volume removed was replaced with an equivalent volume of fresh buffer, the pellets were resuspended, and the tubes were placed back in the Thermomixer. The supernatant samples were added to a 96-well assay plate, and content released from the ALD-coated particles was measured using a Fluoraldehyde o-phthaldialdehyde (OPA) Assay Reagent Mix per the manufacturer's instructions. Fluorescence was measured after excitation at 360 nm and detected at 450 nm using a plate reader. Data can be presented as percent recovery and calculated by normalizing the fluorescent signal against the maximum signal from each sample. Data can be analyzed using four-parameter logistic (4PL) regressions.

All of the compositions and methods disclosed and claimed herein can be made and executed without undue experimentation in light of the present disclosure. While the compositions and methods have been described in terms of embodiments, it is apparent to those of skill in the art that variations may be applied to the compositions and methods and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A dissolution composition comprising:
atomic layer deposition (ALD) coated therapeutic agent-containing particles,
an aqueous composition or buffer,
a pH of about 4.0 to about 10.0; and
a temperature of about 30 °C to about 70 °C.

2. The composition according to claim 1, wherein the ALD coating of the ALD coated therapeutic agent-containing particles comprises a metal-oxide or metal-alkoxide containing composition.

3. The composition according to claim 1 or 2, wherein the ALD coating comprises aluminum oxide, an aluminum alkoxide, silicon dioxide (SiO2), titanium dioxide (TiO2), zinc oxide (ZnO2), zirconium oxide (ZrO₂), silicon nitride (Si3N4), alternating layers, and any combination of metal-oxide and/or metal-alkoxide thereof.

4. The composition according to claim 2 or 3, further comprising wherein the ALD coating comprises layers of a single metal-containing composition, an alternating metal-containing composition of at least two metal-containing compositions, other suitable layering pattern, or layers of a formulation mixture of two or more metal-oxide or metal-alkoxide, or a metal-oxide or metal-alkoxide combined with at least one additional agent for layering on the therapeutic agent-containing particles.

5. The composition according to any one of claims 1-4, wherein the aqueous composition or buffer comprises one or more of distilled water, a phosphate, a citrate, a tris, a nitrate, a borate, a sulfate, a histidine, a HEPES (2-[4-(2-hydroxyethyl)piperazin-1-yl]ethanesulfonic acid), a MES (2-(N-morpholino)ethanesulfonic acid), a MOPS (3-(N-morpholino)propanesulfonic acid), a MOBS (4-(N-Morpholino)butanesulfonic acid), a PIPES (2,2′-piperazine-1,4-diylbisethanesulfonic acid), and a silicic buffer.

6. The composition according to claim 1, wherein the aqueous composition or buffer comprises one or more of distilled water, a phosphate, a citrate, a tris, a nitrate, a borate, a sulfate, a histidine, a HEPES, a MES, a MOPS, a MOBS, a PIPES, and a silicic buffer.

7. The composition according to claim 1, wherein the aqueous composition or buffer comprises a phosphate or a tris buffer.

8. The composition according to any one of claims 1-7, wherein the aqueous composition or buffer comprises a concentration comprising about 0.01 mM to about 2.0 mM, about 1.0 mM to about 1.0. M, or about 10.0 mM to about 1.0 M, or about 50.0 mM to about 500.0 mM, or about 50 mM to about 250.0 mM or about 50 mM to about 200 mM or about 100 mM.

9. The composition according to claim 1, wherein the aqueous composition or buffer comprises a concentration comprising about 0.01 mM to about 2.0 mM, about 1.0 mM to about 1.0. M, or about 10.0 mM to about 1.0 M, or about 50.0 mM to about 500.0 mM, or about 50 mM to about 250.0 mM or about 50 mM to about 200 mM or about 100 mM.

10. The composition according to any one of claims 1-9, wherein the aqueous composition or buffer further comprises a salt-containing aqueous composition or buffer.

11. The composition according to claim 10, wherein the salt comprises at least one of potassium, sodium, magnesium, calcium, copper and the like.

12. The composition according to any one of claims 1-11, wherein the aqueous composition or buffer comprises potassium phosphate.

13. The composition according to claim 1, wherein the aqueous composition or buffer comprises potassium phosphate.

14. The composition according to any one of claims 1-13, wherein pH of the composition comprises a pH of about 5.0 to about 8.0; or about 5.0 to about 7.0 or about 5.5 to about 6.5.

15. The composition according to any one of claims 1-14, wherein temperature of the composition comprises about 35 °C to about 70 °C; or about 40 °C to about 70 °C; or about 40 °C to about 65 °C, or about 40 °C to about 60 °C, or about 40 °C to about 55 °C, or about 45 °C to about 55 °C or about 50 °C.

16. The composition according to any one of claims 1-15, wherein the aqueous composition or buffer is not phosphate buffered saline (PBS) or saline.

17. The composition according to any one of claims 1-16, wherein the therapeutic agents of the ALD coated therapeutic agent-containing particles comprise at least one thermostable therapeutic, prophylactic, or diagnostic agent.

18. The composition according to any one of claims 1-17, wherein a therapeutic agent of the ALD coated therapeutic agent-containing particles can be localized in a single layer or in different layers of the ALD coated therapeutic agent-containing particles for timed-release delivery of the thermostable therapeutic agent.

19. The composition according to claim 18, wherein same or different therapeutic agents can be localized in a single layer or in different layers of the ALD coated therapeutic agent-containing particles for timed delivery of the same or different therapeutic agents.

20. The composition according to any one of claims 1-19, wherein the ALD coated therapeutic agent-containing particles comprise thermostable therapeutic agents encased in a glassy matrix comprising one or more disaccharide agent.

21. The composition according to any one of claims 1-20, wherein a therapeutic agent of the ALD coated therapeutic agent containing particles comprises at least one of polypeptides, proteins, antibody or biologically-relevant fragments thereof, conjugates thereof, polynucleotides (e.g., RNA, DNA, RNA, siRNA or the like), conjugates thereof, oligonucleotides, chimeric molecules, live, attenuated viruses, inactivated viruses, virus-like particles (VLPs), lipopolysaccharides, toxins, small molecules, or other suitable therapeutic agent of use to treat or ameliorate a health condition.

22. The composition according to claim 21, wherein the therapeutic agent comprises a therapeutic agent to reduce the onset of an infection by a pathogenic organism.

23. The method according to claim 1 or 2, wherein the ALD coating comprises aluminum oxide, an aluminum alkoxide, titanium dioxide (TiO2), alternating layers, and any combination thereof.

24. The method according to claim 23, wherein the aqueous composition or buffer comprises one or more of phosphate and citrate buffer.

25. The method according to claim 24, wherein the aqueous composition or buffer comprises one or more of potassium phosphate and sodium citrate buffer.

26. The method according to claim 25, wherein concentration of the composition or buffer is from about 50 mM to about 350 mM, or about 100 mM to about 300 mM.

27. A method for analyzing dissolution of an ALD coated thermostable therapeutic agent-containing particle comprising,
suspending ALD coated therapeutic agent-containing particles in an aqueous composition or buffer having a pH of about 4.0 to about 10.0 to generate an aqueous dissolution composition containing the ALD coated therapeutic agent-containing particles;
exposing the aqueous dissolution composition containing the ALD coated therapeutic agent-containing particles to a temperature of about 30 °C to about 70 °C;
incubating the aqueous composition containing the ALD coated therapeutic agent-containing particles for a pre-determined time; and
separating remaining ALD coated therapeutic agent-containing particles from the aqueous dissolution composition; and
obtaining a sample of the aqueous dissolution composition for analysis of a released therapeutic agent from ALD coated therapeutic agent-containing particles.

28. The method according to claim 27, wherein the method is repeated at least one more time on the separated remaining ALD coated therapeutic agent-containing particles at a pre-determined time.

29. The method of claim 27 or 28, wherein the ALD coated therapeutic agent-containing particles can include at least about 2 to about 1,000 ALD coating layers or more.

30. The method according to any one of claims 27-29, wherein the ALD coating comprises aluminum oxide, an aluminum alkoxide, silicon dioxide (SiO2), titanium dioxide (TiO2), zinc oxide (ZnO2), zirconium oxide (ZrO2), silicon nitride (Si3N4) or other suitable metallo-oxide or metal-alkoxide material and any combination thereof.

31. The method according to any one of claims 27-30, wherein the aqueous composition comprises one or more of distilled water, a phosphate, a citrate, a tris, a nitrate, a borate, a sulfate, a histidine, a HEPES, a MES, a MOPS, a MOBS, a PIPES, and a silicic buffer.

32. The method according to any one of claims 27-31, wherein the aqueous composition comprises phosphate or tris.

33. The method according to any one of claims 27-32, wherein the aqueous composition containing the ALD coated therapeutic agent-containing particles is intermittently or continuously agitated during incubation.

34. The method according to any one of claims 27-33, wherein inflection point of dissolution of a therapeutic agent of the ALD coated therapeutic agent-containing particles can be determined, wherein inflection point comprises a period where peak dissolution and appearance of a therapeutic agent of the ALD coated therapeutic agent-containing particles occurs.

35. The method according to any one of claims 27-34, wherein the method provides consistent, specific, rapid assessment of ALD coat quality and dissolution behavior of a therapeutic agent of the ALD coated therapeutic agent-containing particles compared to real-time analysis to release the thermostable therapeutic agent in vivo.

36. The method according to any one of claims 27-35, wherein a therapeutic agent of the ALD coated therapeutic agent-containing particles comprises at least one therapeutic, prophylactic, or diagnostic agent of use to treat, prevent or ameliorate a health condition.

37. The method according to any one of claims 27-36, wherein a therapeutic agent of the ALD coated therapeutic agent containing particles comprises at least one of polypeptides, proteins, antibody or biologically-relevant fragments thereof, conjugates thereof, polynucleotides (e.g., RNA, DNA, mRNA, siRNA or the like), conjugates thereof, oligonucleotides, chimeric molecules, live, attenuated viruses, inactivated viruses, virus-like particles (VLPs), lipopolysaccharides, toxins, small molecules, or other suitable therapeutic agent of use to treat or ameliorate a health condition.

38. The method according to claim 37, wherein the therapeutic agent comprises a therapeutic agent to reduce the onset of an infection by a pathogenic organism.

39. The method according to any one of claims 27-38, wherein incubating the dissolution composition containing the ALD coated therapeutic agent-containing particles for a pre-determined time comprises incubating for about 15 minutes up to about 3 weeks.

40. A kit comprising:
an aqueous dissolution composition comprising one or more of distilled water, a phosphate, a citrate, a tris, a nitrate, a borate, a sulfate, a histidine, a HEPES, a MES, a MOPS, a MOBS, a PIPES, and a silicic buffer,
at least one of a negative and positive control of at least one ALD coated therapeutic agent or representative therapeutic agent containing particles;
at a pH comprising about 4.0 to about 10.0; and
at least one container.

41. The kit according to claim 40, further comprising a sample of ALD coated thermostable therapeutic agent-containing particles with a predetermined number of ALD coating layers.

42. The kit according to claim 40 or 41, wherein the kit further comprises at least one microtiter plate.

43. The kit according to any one of claims 40-42, wherein the kit further comprises instructions for performing a dissolution assay of ALD coated therapeutic agent-containing particles.
